(19) United States
(12) Patent Application Publication
(10) Pub. No.: US 2023/0346272 A1
(43) Pub. Date: Nov. 2, 2023

(54) ELECTROCHEMICAL SENSOR FOR SIMULTANEOUS DETECTION AND MEASUREMENT OF MULTIPLE PHARMACEUTICALS

(71) Applicant: CARI HEALTH, INC., SAn Diego, CA (US)

(72) Inventors: Rajesh SEENIVASAN, San Diego, CA (US); Mark WECKWERTH, San Diego, CA (US); Torsten FIEBIG, San Diego, CA (US); Patrik SCHMIDLE, San Diego, CA (US)

(21) Appl. No.: 18/026,583

(22) PCT Filed: May 3, 2022
(86) PCT No.: PCT/US2022/027527

§ 371 Date: Mar. 15, 2023

Related U.S. Application Data
(60) Provisional application No. 63/183,440, filed on May 3, 2021.

(51) Int. Cl. A61B 5/1473; C25D 7/00; C25D 3/50; C25D 3/48; A61B 5/145

(52) U.S. Cl. CPC A61B 5/1473; C25D 7/00; C25D 3/50; C25D 3/48; A61B 5/14514; A61B 5/14546; A61B 5/746

(57) ABSTRACT

Provided herein are devices and systems for measuring a dosage of a medication used by a subject. Further provided herein are methods, systems, and media for dosing regulation and prescription compliance.

### CROSS-REFERENCE

This application claims the benefit of U.S. Provisional Application No. 63/183,440 filed May 3, 2021, which is incorporated herein by reference.

### STATEMENT AS TO FEDERALLY SPONSORED RESEARCH

This invention was made with government support under NIH/NIDA Grant #2R44DA044905-02 (FAIN R44DA044905). The government has certain rights in the invention.

### BACKGROUND

Remote medication monitoring (RMM) opens new possibilities for individualized medicine and digital healthcare. Real-time in-vivo drug concentration measurements resolve compliance issues by verifying ingestion of medications and help titrate the right individual doses by assessing metabolization rates for each patient.

### SUMMARY

The present disclosure is generally related to analytical methods, devices, and systems. More specifically, the present disclosure provides analytical methods, devices, and systems for determining the concentration of various analytes, or substances, in the human body.

In an aspect, the present disclosure provides a method of determining the presence or absence of a at least one substance in a bodily fluid of a subject. The method may include a step of attaching the microneedle sensor to the skin of the subject, wherein the microneedle sensor comprises a microneedle array. The method may include a step of applying a sweeping voltage to the microneedle array. The method may include a step of measuring a current, with the microneedle sensor, in response to the sweeping voltage. The method may include a step of determining the presence of absence of the at least one substance in the bodily fluid of the subject using the measured current.

The method may include a step of determining the concentration of the at least one substance in the bodily fluid of the subject using the measured current.

In some embodiments, the bodily fluid may be dermal interstitial fluid.

In some embodiments, the step of determining the presence or absence of the at least one substance in a bodily fluid of a subject may not require an enzymatic reaction involving the at least one substance.

In some embodiments, the step of determining the presence or absence of the at least one substance in a bodily fluid of a subject may not require calibrating the microneedle sensor.

In some embodiments, the method may include repeating the steps of applying, measuring, and determining on an interval between about 1 minute and about 6 hours.

In some embodiments, the microneedle sensor may be attached to the skin for about 1 day to about 30 days. In some embodiments, the microneedle sensor may be attached to the skin for at least about 30 days without substantial biofouling.

In some embodiments, the sweeping voltage may be from about −0.7 V to about +1.5 V. In some embodiments, the sweeping voltage may include a single staircase voltage sweep. In some embodiments, the single staircase voltage sweep may occur over a period of about 5 seconds to about 5 minutes. In some embodiments, the sweeping voltage may include multiple staircase voltage sweeps. In some embodiments, the multiple staircase voltage sweeps may occur over a period of about 5 seconds to about 5 minutes.

In some embodiments, the microneedle array may include at least one working electrode, at least one reference electrode, at least one counter electrode, and a potentiostat. In some embodiments, the at least one working electrode, at least one reference electrode, and the at least one counter electrode each independently may include at least one microneedle of the microneedle array.

In some embodiments, the step of applying the sweeping voltage to the microneedle array may include applying the sweeping voltage between the at least one working electrode and the at least one reference electrode.

In some embodiments, the step of measuring the current may include generating at least one voltammogram. In some embodiments, the at least one voltammogram may be at least one DPV voltammogram.

In some embodiments, the presence of absence of the at least one substance in the bodily fluid of the subject may be determined using the at least one voltammogram. In some embodiments, the presence of absence of the at least one substance in the bodily fluid of the subject may be determined using the at least one DPV voltammogram.

In some embodiments, the presence of absence of the at least one substance in the bodily fluid of the subject may be determined using at least one voltage peak of the at least one voltammogram. In some embodiments, the presence of absence of the at least one substance in the bodily fluid of the subject may be determined using at least one voltage peak of the at least one DPV voltammogram.

In some embodiments, the at least one substance in the bodily fluid of the subject may be present when the measured current is above a threshold.

In some embodiments, the step of determining the presence of absence of the at least one substance in the bodily fluid of the subject may include determining the concentration of the at least one substance.

In some embodiments, the at least one substance in the bodily fluid of the subject may be present when the concentration of the at least one substance is above a threshold.

In some embodiments, the step of determining the presence or absence of the at least one substance in the bodily fluid of the subject may include determining the presence or absence of at least two substances in the bodily fluid of the subject concurrently using the measured current.

In some embodiments, the measured concentration of the substance may be time-dependent.

In some embodiments, the at least one substance may be a pharmaceutical compound or a metabolite of the pharmaceutical compound.

In some embodiments, pharmaceutical compound or a metabolite of the pharmaceutical compound may include methadone, 2-ethylidene-1,5-dimethyl-3,3-diphenylpyrrolidine (EDDP), methadone-3-glucuronide, methadone-6-glucuronide, buprenorphine or a buprenorphine metabolite is selected from buprenorphine, norbuprenorphine, buprenorphine-3-glucuronide, norbuprenorphine-3-glucuronide, codeine, dihydrocodeine, fentanyl, carfentanyl, hydromorphone, hydrocodone, meperidine, morphine, oxycodone, oxymorphone, tapentadol, tramadol, methotrexate, gemcitabine, 6-mercaptopurine, fludarabine, cytarabine, pemetrexed, cyclosporines, mycophenolate mofetil, prednisone, tacrolimus, azathioprine, ciprofloxacin, amoxicillin, vancomycin, gentamycin, tetracyclines, cannabidiol, alprazolam, diazepam, lorazepam, chlordiazepoxide, clonazepam, midazolam, clobazam, gabapentin, pregabalin, oxcarbazepine, phenobarbital, valproic acid, zonisamide, levetiracetam, lamotrigine, lacosamide, topiramate, fluoxetine, sertraline, paroxetine, escitalopram, citalopram, dioxin, digitoxin, flecainide, lithium, phenytoin, rifampicin, theophylline, warfarin, sodium, potassium, iron, cobalt, copper, magnesium, manganese, norepinephrine, epinephrine, testosterone, estrogen, progesterone, cortisol, aldosterol, dopamine, serotonin, acetylcholine, creatinine, urea, uric acid, glucose, glutamine, lactate, amino acids, amino acid metabolites, vitamins, essential fatty acids, ammonia, insulin, transferrin, insulin-like growth factor I and II, human platelet-derived growth factor I, human platelet-derived growth factor II, fibroblasts growth factors, tissue specific growth factors, acetaminophen, alfentanil, amiodipine, amitriptyline, amprenavir, atorvastatin, bepridil, buspirone, caffeine, carbamazepine, carvedilol, celecoxib, cerivastatin, chlorpromazine, cisapride, citalopram, clarithromycin, clomipramine, clozapine, corticosteroids, cyclobenzaprine, cyclophosphamide, dapsone, delavirdine, desipramine, dextromethorphan, diclofenac, diltiazem, disopyramide, dofetilide, donepezil, doxorubicin, efavirenz, erythromycin, ethinylestradiol, etoposide, felodipine, finasteride, flecainide, flurbiprofen, fluvastatin, glimepiride, grepafloxacin, haloperidol, ibuprofen, ifosfamide, imipramine, indinavir, irbesartan, isradipine, itraconazole, ketoconazole, lansoprazole, loratadine, losartan, lovastatin, maprotiline, metoprolol, mexiletine, mirtazapine, montelukast, naproxen, nefazodone, nelfinavir, nicardipine, nifedipine, nimodipine, nortriptyline, olanzapine, omeprazole, paclitaxel, paroxetine, perphenazine, pimozide, piroxicam, propafenone, propranolol, propranolol, propofol, quetiapine, quinidine, quinine, rifabutin, repaglinide, riluzole, risperidone, ritonavir, ropinirole, saquinavir, sertraline, sibutamine, sildenafil, simvastatin, sirolimus, sufentanil, tacrine, tacrolimus, tamoxifen, testosterone, theophylline, thoridazine, timolo, tolbutamide, tolterodine, toremifene, torsemide, trazodone, triazolam, troleandomycin, venlafaxine, verapamil, vinblastine, vincristine, zafirlukast, zaleplon, zileuton, zolpidem, and combinations thereof.

In some embodiments, the method may further include the step of transmitting, with the microneedle sensor, a signal generated using the determined presence or absence of the at least one substance in the bodily fluid of the subject, the measured current, or both, to one or more of an external device, system or network.

In some embodiments, the method may further include the step of transmitting, with the microneedle sensor, a signal generated using the determined concentration of the at least one substance in the bodily fluid of the subject.

In some embodiments, the signal may be transmitted periodically at about 1 min to about 6-hour intervals.

In some embodiments, the signal may be transmitted to the one or more external device, system, or network when the absence of the at least one substance is determined.

In some embodiments, the signal may be transmitted to the one or more external device, system, or network when the concentration of the at least one substance is below a threshold concentration.

In another aspect, the present disclosure provides a sensor device for determining the presence or absence of at least one substance in a bodily fluid of a subject. In some embodiments, the device may include a body configured to attach the skin of the subject. In some embodiments, the device may include a microneedle array coupled to the body. In some embodiments, the device may include a control module disposed within the body. In some embodiments, the control module may include a potentiostat coupled to the microneedle array, a processor coupled to the potentiostat, and a memory coupled to the processor. In some embodiments, the memory may include instructions for the processor to perform the methods described herein.

In another aspect, the present disclosure provides a sensor device for determining the presence or absence of at least one substance in a bodily fluid of a subject. In some embodiments, the device may include a body configured to attach the skin of the subject. In some embodiments, the device may include a microneedle array coupled to the body. In some embodiments, the device may include a control module disposed within the body. In some embodiments, the control module may include a potentiostat coupled to the microneedle array, a processor coupled to the potentiostat, and a memory coupled to the processor, In some embodiments, the memory may include instructions for the processor to: apply a sweeping voltage to the microneedle array; measure a current in response to applying the sweeping voltage; and determine the presence or absence of the at least one substance in the bodily fluid of the subject using the measured current.

In some embodiments, body may include an adhesive.

In some embodiments, at least one microneedle of the microneedle array may be plated with gold, platinum, silver, silver chloride, or combinations thereof.

In some embodiments, at least one microneedle of the microneedle array may be coated with an electrode coating.

In some embodiments, the electrode coating may include a quaternary ammonium, sulfonated tetrafluoroethylene, polyethyleneimine-functionalized carbon nanotube, or combinations thereof.

In some embodiments, the microneedle array may include at least one working electrode and at least one reference electrode.

In some embodiments, the sweeping voltage may be applied between the at least one working electrode and the at least one reference electrode.

In some embodiments, the device may not include an enzymatic layer.

In some embodiments, the device may not include a microfluidic element, microfluidic channel, or microfluidic pump.

In some embodiments, the memory may further include instructions for the processor to determine the presence or absence of at least two substances in the bodily fluid of the subject concurrently using the measured current.

In some embodiments, the device may be configured to be attached to the skin for about 1 day to about 30 days.

In some embodiments, the device may be configured to be attached to the skin for at least about 30 days without biofouling.

In some embodiments, the device may further include a receiver coupled to the processor.

In some embodiments, the device may further include a transmitter coupled to the processor.

In some embodiments, the transmitter may be configured to transmit a signal based on the determined presence or absence of the at least one substance, the measured current, or both, to one or more of an external device, system, or network. In some embodiments, the signal may be based on a measured concentration of the at one substance.

In some embodiments, the memory may further include instructions to transmit the signal periodically at about 1 min to about 6-hour intervals.

In some embodiments, the one or more of an external device, system, or network may include one or more of a smart phone, tablet computer, personal computer, gaming console, workstation, network computer, or network server.

In some embodiments, the one or more of an external device, system, or network may include a memory including instructions for the one or more of an external device, system, or network to operate an application including one or more of a measurement display or a user control for the sensor device. In some embodiments, the application may be configured to provide an alert to a user based on the determined presence or absence of the at least one substance in the bodily fluid of a subject. In some embodiments, the application may be configured to provide an alert to a caregiver based on the determined presence or absence of the at least one substance in the bodily fluid of a subject. In some embodiments, a caregiver may be selected from the group consisting of physician, clinician, pharmacy, government enforcement agency or combinations thereof.

In some embodiments, the device may further include an alarm configured to generate an alert to a user based on the determined presence or absence of the at least one substance, the measured current, or both. In some embodiments, the device may further include an alarm configured to generate an alert to a caregiver based on the determined presence or absence of the at least one substance, the measured current, or both. In some embodiments, a caregiver may be selected from the group consisting of physician, clinician, pharmacy, government enforcement agency or combinations thereof.

In another aspect, the present disclosure provides a system for determining the presence or absence of at least one substance in a bodily fluid of a subject. In some embodiments, the system may include an external device, system, or network. In some embodiments, the system may include the device described herein. In some embodiments, the device may further include a transmitter coupled to a processor of the device. In some embodiments, the transmitter may be configured to transmit a signal based on the determined presence or absence of the at least one substance, the measured current, or both to the external device, system, or network. In some embodiments, the external device, system, or network may include a memory including instructions for the external device, system, or network to operate an application including a measurement display or a user control for the device.

In another aspect, the present disclosure provides a microneedle array. In some embodiments, the microneedle array may include a plurality of microneedles. In some embodiments, the microneedle array may include a metal or metal alloy plating for the plurality of microneedles. In some embodiments, the microneedle array may include an electroactive cage material deposited over the metal plating. In some embodiments, the electroactive cage material may include an electroactive composite. In some embodiments, the electroactive composite may include multiwall carbon nanotubes and a polyethyleneimine polymer. In some embodiments, the electroactive cage material may include a stabilizing polymer matrix embedding and surrounding the electroactive composite. In some embodiments, the microneedle array may include a hydrogel coating provided over the electroactive cage.

In some embodiments, the metal plating may include gold, platinum, silver, silver chloride, or combinations thereof.

In some embodiments, the stabilizing polymer matrix may include an alkyl ammonium salt and a sulfonated tetrafluoroethylene polymer.

In some embodiments, the hydrogel coating may include an agarose hydrogel.

In some embodiments, the hydrogel coating may be doped with potassium chloride.

In some embodiments, the hydrogel coating may be configured to protect the electroactive cage material from the deposition of molecular redox byproducts.

In another aspect, the present disclosure provides a method of processing a microneedle array. In some embodiments, the method may include plating as metal or metal alloy onto a plurality of microneedles. In some embodiments, the method may include depositing an electroactive cage material over the metal coating. In some embodiments, the electroactive cage material may include an electroactive composite. In some embodiments, the electroactive composite may include multiwall carbon nanotubes and a polyethyleneimine polymer; In some embodiments, the electroactive cage material may include a stabilizing polymer matrix bound to the electroactive composite. In some embodiments, the method may include providing a hydrogel coating over the electroactive cage material on the plurality of microneedles.

In some embodiments, the metal plating may include gold, platinum, silver, silver chloride, and combinations thereof.

In some embodiments, the method may further include the step of pre-treating the metal or metal alloy plated plurality of microneedles prior to depositing the electro active cage material.

In some embodiments, the step of pre-treating the metal or metal alloy plated plurality of microneedles may include applying an acid to the metal or metal alloy plated plurality of microneedles. In some embodiments, the step of pre-treating the metal or metal alloy plated plurality of microneedles may include applying a linear potential sweep between the metal or metal alloy plated plurality of microneedles and a reference electrode. In some embodiments, the step of pre-treating the metal or metal alloy plated plurality of microneedles may include washing the metal or metal alloy plated plurality of microneedles with de-ionized water. In some embodiments, the step of pre-treating the metal or metal alloy plated plurality of microneedles may include drying the metal or metal alloy plated plurality of microneedles.

In some embodiments, the method may further include the step of producing the electroactive cage by dispersing the multiwall carbon nanotubes into a solution to generate a dispersion and combining the dispersion with an aqueous solution comprising the polyethyleneimine polymer.

In some embodiments, the method may further include the step of removing unbound multiwall carbon nanotubes and polyethyleneimine polymer.

In some embodiments, the method may further include the step of drying the composite after the unbound multiwall carbon nanotubes and polyethyleneimine polymer is removed.

In some embodiments, the method may further include the step of combining the electroactive composite and the stabilizing polymer matrix and allowing the electroactive composite to self-assemble into the electroactive cage material.

In some embodiments, the stabilizing polymer matrix may include an alkyl ammonium salt and a sulfonated tetrafluoroethylene polymer.

In some embodiments, the step of depositing the electroactive cage material over the metal coating may include drop-casting the electroactive cage material.

In some embodiments, the hydrogel coating may include an agarose hydrogel.

In some embodiments, the hydrogel coating may be doped with potassium chloride.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1 is block diagram illustrating an exemplary system for the detection and measurement of various analytes, according to some embodiments.

FIG. 2 illustrates an exploded view of a stylized rendering of the hardware components of an exemplary sensor, according to some embodiments.

FIG. 3 is an illustration depicting the cross section of an exemplary microneedle array having a contact surface comprising a plurality of needles, according to some embodiments.

FIG. 4 shows a non-limiting example of a computing device; in this case, a device with one or more processors, memory, storage, and a network interface.

FIG. 5 shows a non-limiting example of a web/mobile application provision system; in this case, a system providing browser-based and/or native mobile user interfaces

FIG. 6 shows a non-limiting example of a cloud-based web/mobile application provision system; in this case, a system comprising an elastically load balanced, auto-scaling web server and application server resources as well synchronously replicated databases.

FIG. 7A is a diagram of an exemplary system comprising a sensor, a computer system, and a cloud-based architecture, for the measurement of analytes in the bodily fluid of a subject, according to some embodiments.

FIG. 7B is an illustration of an exemplary application and user interface, according to some embodiments.

FIG. 8 illustrates a method for processing a microneedle array, according to some embodiments.

FIG. 9 is a flow chart illustrating an application for the exemplary system for simultaneous measurement of multiple analytes, according to some embodiments.

FIG. 10 is a graph of an exemplary voltammogram graph for measurement of oxycodone and its two main metabolites in humans: noroxycodone and oxymorphone, according to some embodiments.

FIG. 11A is a graph of an exemplary DPV graph for measurement of buprenorphine, according to some embodiments.

FIG. 11B is a graph of an exemplary DPV graph for measurement of norbuprenorphine, according to some embodiments.

FIG. 11C is a graph of an exemplary DPV graph for measurement of buprenorphine and norbuprenorphine, according to some embodiments.

FIG. 12 is a chart showing the voltages at which the DPV curves peak for various substances, according to some embodiments.

FIG. 13 is a graph of an exemplary DPV for daily measurement of an oxycodone standard solution with fixed concentration of 1.0 μg/mL for two sensors over a time period of 15 days, according to some embodiments.

FIG. 14A is a graph showing DPV scans for methadone solution in PBS at various concentrations, according to some embodiments.

FIG. 14B is a graph or a calibration curve demonstrating a linear relationship between the methadone concentration and the peak height (in μA), according to some embodiments.

FIG. 15 is a graph showing DPV scans (in PBS solution) of methadone, methadone's main metabolite, EDDP, and a mixture of both substances, according to some embodiments.

### DETAILED DESCRIPTION

Provided herein are systems, devices, and methods for determining the presence and concentration of specific analytes in the dermal interstitial fluid of a subject.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Definitions

Unless defined otherwise, all terms of art, notations, and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this disclosure, various embodiments may be presented in a range of formats. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, a description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including combinations thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative, or quantitative and qualitative determinations. Assessing can be relative or absolute. “Detecting the presence of” can include determining the amount of something present in addition to determining whether it is present or absent depending on the context.

The term “about” or “approximately” as used herein when referring to a measurable value such as an amount or concentration and the like, is meant to encompass variations of 20%, 10%, 5%, 1%, 0.5%, or even 0.1% of the specified amount. For example, “about” can mean plus or minus 10%, per the practice in the art. Alternatively, “about” can mean a range of plus or minus 20%, plus or minus 10%, plus or minus 5%, or plus or minus 1% of a given value. Alternatively, particularly with respect to biological systems or processes, the term can mean within an order of magnitude, up to 5-fold, or up to 2-fold, of a value. Where particular values can be described in the application and claims, unless otherwise stated the term “about” may be assumed to encompass the acceptable error range for the particular value. Also, where ranges, subranges, or both, of values can be provided, the ranges or subranges can include the endpoints of the ranges or subranges.

Where values are described as ranges, it may be understood that such disclosure includes the disclosure of all possible sub-ranges within such ranges, as well as specific numerical values that fall within such ranges irrespective of whether a specific numerical value or specific sub-range is expressly stated.

The terms “comprise,” “have,” and “include” are open-ended linking verbs. Any forms or tenses of one or more of these verbs, such as “comprises,” “comprising,” “has,” “having,” “includes,” and “including,” are also open-ended. For example, any method that “comprises,” “has,” or “includes” one or more steps is not limited to possessing only those one or more steps and also covers other unlisted steps.

As used herein, the term “mixture” or “mixtures” is meant to include a simple combination of materials and any compounds that may result from their combination.

The section headings used herein are for organizational purposes only and are not to be construed as limiting the subject matter described.

### Remote Medication Monitoring Systems

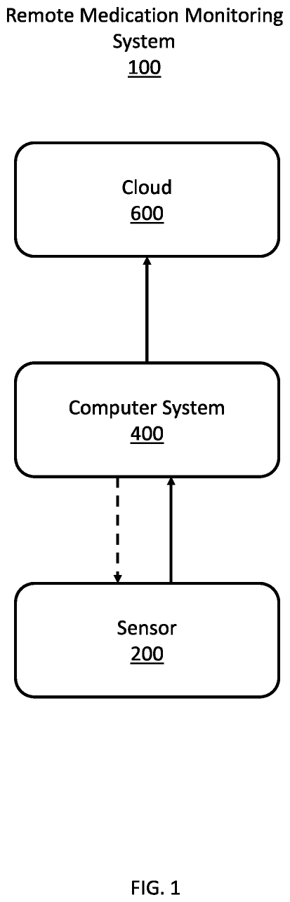

In some embodiments, described herein are Remote Medication Monitoring (RMM) system. FIG. 1. illustrates a remote medication monitoring system, according some embodiments. The remote medication monitoring system 100 may comprise a sensor 200, a computer system 400, and a cloud network architecture 600. In some embodiments, the RMM may detect the presence, absence, or concentration of an analyte in the bodily fluids of a subject. In some embodiments, the RMM may transmit by the monitor system to the patient, the patient's caregivers, the patient's clinicians, the patient's family members, or any combination thereof. In some embodiments, caregivers may be selected from a physician, a clinician, a nurse practitioner, a hospital, a clinic, a rehabilitation facility, a pharmacy, a designated family caregiver, or a legal or healthcare enforcement agency. In some embodiments, the measured concentration of the substance is received by a computer system of the patient, the patient's caregivers, the patient's clinicians, the patient's family members, or any combination thereof. In some embodiments, the computer system may be a smartphone. In some embodiments, the data is being transferred to an application which may apply additional data processing. In some embodiments, the application is a smartphone application. In some embodiments, the measured concentration of the substance is analyzed to determine patterns, recommend improvements, or both. In some embodiments, the RMM enables detection and/or quantitation of analytes in-vivo (i.e., without a need to sample bodily fluids and analyze them externally).

### Sensor

The RMM systems described herein may comprise a sensor 200. The sensor 200, may be a wearable sensor. The sensor 200 may be configured to detect an analyte in the bodily fluid of a subject.

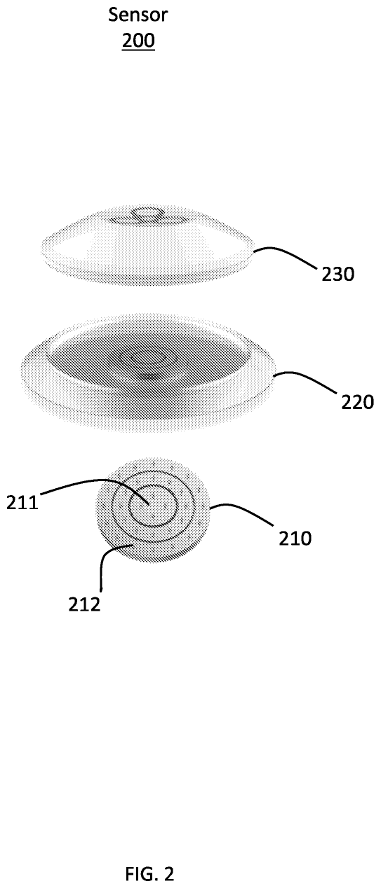

FIG. 2 illustrates a sensor 200, according to some embodiments. The sensor 200 may comprise a body 220, a microneedle array 210 coupled to the body, and a control module 230 (also referred to herein as a “COM module”) disposed within the body 220 and operably coupled to the microneedle array 210.

In some embodiments, the microneedle array 210 may be a rigid array. In some embodiments, the microneedle array 210, may be made of a material selected from a metal, a silicon material, a polymeric material, and combinations thereof. In some embodiments, the microneedle array 210 may be made of a metal selected from gold, silver, platinum, copper, palladium, nickel, iridium, rhodium, and cobalt. In some embodiments, the microneedle array may be made of a metal selected from gold, platinum, silver, or combinations thereof. In some embodiments, the microneedle array 210 may be made of silver.

In some embodiments, the microneedle array 210 is made of a conductive polymer or a non-conductive polymer plated with a conductive material such as a metal.

In some embodiments, the microneedle array 210 comprises a contact surface 211 adapted to contact the skin of a subject. In some embodiments, the contact surface 211 may have a shape selected from triangular, square, circular, and polygonal (e.g., rectangular, hexagonal, octagonal, etc.).

In some embodiments. each dimension of the contact surface 211 of the microneedle array 210 is independently about 2 mm to about 20 mm. In some embodiments, each dimension of the contact surface 211 is independently about 2 mm to about 3 mm, about 2 mm to about 5 mm, about 2 mm to about 7 mm, about 2 mm to about 10 mm, about 2 mm to about 12 mm, about 2 mm to about 15 mm, about 2 mm to about 17 mm, about 2 mm to about 20 mm, about 3 mm to about 5 mm, about 3 mm to about 7 mm, about 3 mm to about 10 mm, about 3 mm to about 12 mm, about 3 mm to about 15 mm, about 3 mm to about 17 mm, about 3 mm to about 20 mm, about 5 mm to about 7 mm, about 5 mm to about 10 mm, about 5 mm to about 12 mm, about 5 mm to about 15 mm, about 5 mm to about 17 mm, about 5 mm to about 20 mm, about 7 mm to about 10 mm, about 7 mm to about 12 mm, about 7 mm to about 15 mm, about 7 mm to about 17 mm, about 7 mm to about 20 mm, about 10 mm to about 12 mm, about 10 mm to about 15 mm, about 10 mm to about 17 mm, about 10 mm to about 20 mm, about 12 mm to about 15 mm, about 12 mm to about 17 mm, about 12 mm to about 20 mm, about 15 mm to about 17 mm, about 15 mm to about 20 mm, or about 17 mm to about 20 mm. In some embodiments, each dimension of the contact surface 211 is independently about 2 mm, about 3 mm, about 5 mm, about 7 mm, about 10 mm, about 12 mm, about 15 mm, about 17 mm, or about 20 mm. In some embodiments, each dimension of the contact surface 211 is independently at least about 2 mm, about 3 mm, about 5 mm, about 7 mm, about 10 mm, about 12 mm, about 15 mm, or about 17 mm. In some embodiments, each dimension of the contact surface 211 independently at most about 3 mm, about 5 mm, about 7 mm, about 10 mm, about 12 mm, about 15 mm, about 17 mm, or about 20 mm.

In some embodiments, the contact surface 211 may comprise a plurality of needles 212 oriented perpendicular to the contact surface 211 and adapted to penetrate the stratum corneum, epidermis, dermal-epidermal (DE) junction, and/or dermis of a subject. In some embodiments, the plurality of needles 212 may be adapted to penetrate the epidermis and descend into the superficial dermis to a depth of about 50 μm to about 100 μm less than the length of the needle. In some embodiments, the plurality of needles 212 may be adapted to provide physical access to the dermal interstitium (the liquid-filled space between skin cells).

In some embodiments, each needle of the plurality of needles 212 may each independently have a cross-sectional shape selected from square pyramidal, conical, and polygonal (e.g. tapered or straight walled triangular, rectangular, hexagonal, octagonal, etc.).

In some embodiments, the length of each needle of the plurality of needles 212 on the contact surface 211 is independently about 600 μm to about 1000 μm. In some embodiments, the length of each needle of the plurality of needles 212 is independently about 600 μm to about 620 μm, about 600 μm to about 640 μm, about 600 μm to about 660 μm, about 600 μm to about 680 μm, about 600 μm to about 700 μm, about 600 μm to about 720 μm, about 600 μm to about 740 μm, about 600 μm to about 760 μm, about 600 μm to about 780 μm, about 600 μm to about 800 μm, about 600 μm to about 820 μm, about 620 μm to about 640 μm, about 620 μm to about 660 μm, about 620 μm to about 680 μm, about 620 μm to about 700 μm, about 620 μm to about 720 μm, about 620 μm to about 740 μm, about 620 μm to about 760 μm, about 620 μm to about 780 μm, about 620 μm to about 800 μm, about 620 μm to about 820 μm, about 640 μm to about 660 μm, about 640 μm to about 680 μm, about 640 μm to about 700 μm, about 640 μm to about 720 μm, about 640 μm to about 740 μm, about 640 μm to about 760 μm, about 640 μm to about 780 μm, about 640 μm to about 800 μm, about 640 μm to about 820 μm, about 660 μm to about 680 μm, about 660 μm to about 700 μm, about 660 μm to about 720 μm, about 660 μm to about 740 μm, about 660 μm to about 760 μm, about 660 μm to about 780 μm, about 660 μm to about 800 μm, about 660 μm to about 820 μm, about 680 μm to about 700 μm, about 680 μm to about 720 μm, about 680 μm to about 740 μm, about 680 μm to about 760 μm, about 680 μm to about 780 μm, about 680 μm to about 800 μm, about 680 μm to about 820 μm, about 700 μm to about 720 μm, about 700 μm to about 740 μm, about 700 μm to about 760 μm, about 700 μm to about 780 μm, about 700 μm to about 800 μm, about 700 μm to about 820 μm, about 720 μm to about 740 μm, about 720 μm to about 760 μm, about 720 μm to about 780 μm, about 720 μm to about 800 μm, about 720 μm to about 820 μm, about 740 μm to about 760 μm, about 740 μm to about 780 μm, about 740 μm to about 800 μm, about 740 μm to about 820 μm, about 760 μm to about 780 μm, about 760 μm to about 800 μm, about 760 μm to about 820 μm, about 780 μm to about 800 μm, about 800 μm to about 840 μm, about 820 μm to about 860 μm, about 840 μm to about 880 μm, about 860 μm to about 900 μm, about 880 μm to about 920 μm, about 900 μm to about 940 μm, about 920 to about 960 μm, about 940 μm to about 980 or about 960 μm to about 1000 In some embodiments, the length of each needle of the plurality of needles 212 is independently about 600 μm, about 620 μm, about 640 μm, about 660 μm, about 680 μm, about 700 μm, about 720 μm, about 740 μm, about 760 μm, about 780 μm, about 800 μm, about 820 μm, about 840 μm, about 860 μm, about 880 μm, about 900 μm, about 920 μm, about 940 μm, about 960 μm, about 980 or about 1000 μm. In some embodiments, the length of each needle of the plurality of needles 212 is independently at least about 600 μm, about 620 μm, about 640 μm, about 660 μm, about 680 μm, about 700 μm, about 720 μm, about 740 μm, about 760 μm, about 780 μm, about 800 μm, about 820 μm, about 840 μm, about 860 μm, about 880 μm, about 900 μm, about 920 μm, about 940 μm, about 960 μm, about 980 μm. In some embodiments, the length of each needle of the plurality of needles 212 is independently at most about 620 μm, about 640 μm, about 660 μm, about 680 μm, about 700 μm, about 720 μm, about 740 μm, about 760 μm, about 780 μm, about 800 μm, about 820 μm, about 840 μm, about 860 μm, about 880 μm, about 900 μm, about 920 μm, about 940 μm, about 960 μm, about 980 μm, or about 1000 μm.

In some embodiments, each needle of the plurality of needles 212 on the contact surface 211 may independently have either straight or tapered walls. In some embodiments, each needle of the plurality of needles 212 may be characterized by their width or diameter at their base.

In some embodiments, the width or diameter at the base of each needle of the plurality of needles 212 on the contact surface 211 is independently about 50 μm to about 300 In some embodiments, the width or diameter at the base each needle of the plurality of needles 212 is independently about 50 μm to about 75 μm, about 50 μm to about 100 μm, about 50 μm to about 120 μm, about 50 μm to about 140 μm, about 50 μm to about 160 μm, about 50 μm to about 180 μm, about 50 μm to about 200 μm, about 50 μm to about 220 μm, about 50 μm to about 240 μm, about 50 μm to about 250 μm, about 50 μm to about 300 μm, about 75 μm to about 100 μm, about 75 μm to about 120 μm, about 75 μm to about 140 μm, about 75 μm to about 160 μm, about 75 μm to about 180 μm, about 75 μm to about 200 μm, about 75 μm to about 220 μm, about 75 μm to about 240 μm, about 75 μm to about 250 μm, about 75 μm to about 300 μm, about 100 μm to about 120 μm, about 100 μm to about 140 μm, about 100 μm to about 160 μm, about 100 μm to about 180 μm, about 100 μm to about 200 μm, about 100 μm to about 220 μm, about 100 μm to about 240 μm, about 100 μm to about 250 μm, about 100 μm to about 300 μm, about 120 μm to about 140 μm, about 120 μm to about 160 μm, about 120 μm to about 180 μm, about 120 μm to about 200 μm, about 120 μm to about 220 μm, about 120 μm to about 240 μm, about 120 μm to about 250 μm, about 120 μm to about 300 μm, about 140 μm to about 160 μm, about 140 μm to about 180 μm, about 140 μm to about 200 μm, about 140 μm to about 220 μm, about 140 μm to about 240 μm, about 140 μm to about 250 μm, about 140 μm to about 300 μm, about 160 μm to about 180 μm, about 160 μm to about 200 μm, about 160 μm to about 220 μm, about 160 μm to about 240 μm, about 160 μm to about 250 μm, about 160 μm to about 300 μm, about 180 μm to about 200 μm, about 180 μm to about 220 μm, about 180 μm to about 240 μm, about 180 μm to about 250 μm, about 180 μm to about 300 μm, about 200 μm to about 220 μm, about 200 μm to about 240 μm, about 200 μm to about 250 μm, about 200 μm to about 300 μm, about 220 μm to about 240 μm, about 220 μm to about 250 μm, about 220 μm to about 300 μm, about 240 μm to about 250 μm, about 240 μm to about 300 or about 250 μm to about 300 In some embodiments, the width or diameter at the base of each needle of the plurality of needles 212 is independently about 50 μm, about 75 μm, about 100 μm, about 120 μm, about 140 μm, about 160 μm, about 180 μm, about 200 μm, about 220 μm, about 240 μm, about 250 or about 300 In some embodiments, the width or diameter at the base of each needle of the plurality of needles 212 is independently at least about 50 μm, about 75 μm, about 100 μm, about 120 μm, about 140 μm, about 160 μm, about 180 μm, about 200 μm, about 220 μm, about 240 or about 250 In some embodiments, the width or diameter at the base of each needle of the plurality of needles 212 is independently at most about 75 μm, about 100 μm, about 120 μm, about 140 μm, about 160 μm, about 180 μm, about 200 μm, about 220 μm, about 240 μm, about 250 or about 300 μm.

In some embodiments, the pitch of the array may describe the distance between adjacent needles of the plurality of needles 212 on the contact surface 211. In some embodiments, the plurality of needles 212 is uniformly spaced. In some embodiments, the plurality of needles 212 is non-uniformly spaced.

In some embodiments, the pitch of the array is about 0.5 mm to about 5 mm. In some embodiments, the pitch of the array is about 0.5 mm to about 1 mm, about 0.5 mm to about 1.5 mm, about 0.5 mm to about 2 mm, about 0.5 mm to about 2.5 mm, about 0.5 mm to about 3 mm, about 0.5 mm to about 3.5 mm, about 0.5 mm to about 4 mm, about 0.5 mm to about 4.5 mm, about 0.5 mm to about 5 mm, about 1 mm to about 1.5 mm, about 1 mm to about 2 mm, about 1 mm to about 2.5 mm, about 1 mm to about 3 mm, about 1 mm to about 3.5 mm, about 1 mm to about 4 mm, about 1 mm to about 4.5 mm, about 1 mm to about 5 mm, about 1.5 mm to about 2 mm, about 1.5 mm to about 2.5 mm, about 1.5 mm to about 3 mm, about 1.5 mm to about 3.5 mm, about 1.5 mm to about 4 mm, about 1.5 mm to about 4.5 mm, about 1.5 mm to about 5 mm, about 2 mm to about 2.5 mm, about 2 mm to about 3 mm, about 2 mm to about 3.5 mm, about 2 mm to about 4 mm, about 2 mm to about 4.5 mm, about 2 mm to about 5 mm, about 2.5 mm to about 3 mm, about 2.5 mm to about 3.5 mm, about 2.5 mm to about 4 mm, about 2.5 mm to about 4.5 mm, about 2.5 mm to about 5 mm, about 3 mm to about 3.5 mm, about 3 mm to about 4 mm, about 3 mm to about 4.5 mm, about 3 mm to about 5 mm, about 3.5 mm to about 4 mm, about 3.5 mm to about 4.5 mm, about 3.5 mm to about 5 mm, about 4 mm to about 4.5 mm, about 4 mm to about 5 mm, or about 4.5 mm to about 5 mm. In some embodiments the pitch of the array is about 0.5 mm, about 1 mm, about 1.5 mm, about 2 mm, about 2.5 mm, about 3 mm, about 3.5 mm, about 4 mm, about 4.5 mm, or about 5 mm. In some embodiments, the pitch of the array is at least about 0.5 mm, about 1 mm, about 1.5 mm, about 2 mm, about 2.5 mm, about 3 mm, about 3.5 mm, about 4 mm, or about 4.5 mm. In some embodiments, the pitch of the array is at most about 1 mm, about 1.5 mm, about 2 mm, about 2.5 mm, about 3 mm, about 3.5 mm, about 4 mm, about 4.5 mm, or about 5 mm.

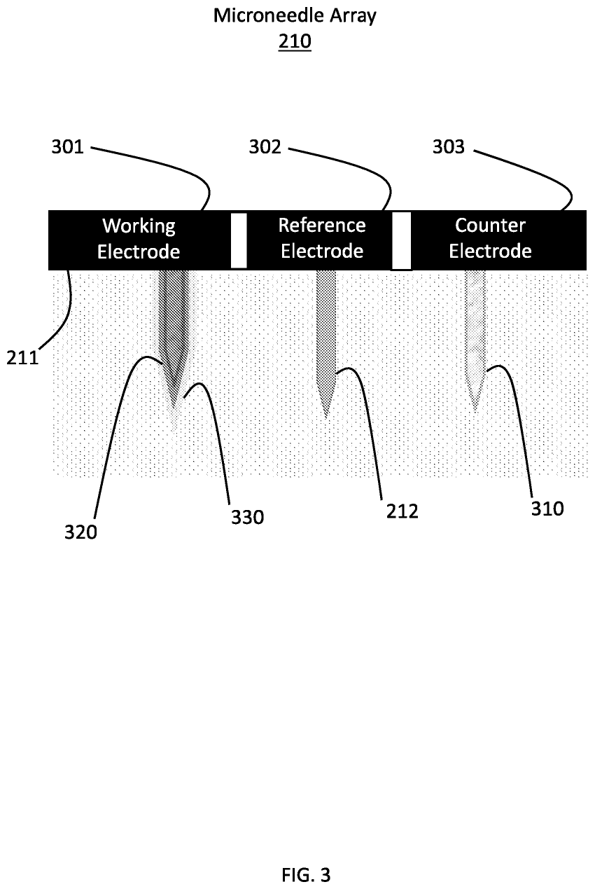

FIG. 3 is an illustration depicting the cross section of the microneedle array 210 having a contact surface 211 comprising a plurality of needles 212.

In some embodiments, each needle of the plurality of needles 212 on the contact surface 211 may comprise a metal plating 310. In some embodiments, the metal plating 310 may comprise gold, platinum, silver, or combinations thereof. In some embodiments, the metal plating 310 may be silver chloride.

In some embodiments the total thickness of the metal plating 310 on each needle of the plurality of needles 212 is about 5 μm to about 25 μm. In some embodiments the total thickness of the metal plating 310 is about 5 μm to about 7 μm, about 5 μm to about 9 μm, about 5 μm to about 11 μm, about 5 μm to about 13 μm, about 5 μm to about 15 μm, about 5 μm to about 17 μm, about 5 μm to about 19 μm, about 5 μm to about 21 μm, about 5 μm to about 23 μm, about 5 μm to about 25 μm, about 7 μm to about 9 μm, about 7 μm to about 11 μm, about 7 μm to about 13 μm, about 7 μm to about 15 μm, about 7 μm to about 17 μm, about 7 μm to about 19 μm, about 7 μm to about 21 μm, about 7 μm to about 23 μm, about 7 μm to about 25 μm, about 9 μm to about 11 μm, about 9 μm to about 13 μm, about 9 μm to about 15 μm, about 9 μm to about 17 μm, about 9 μm to about 19 μm, about 9 μm to about 21 μm, about 9 μm to about 23 μm, about 9 μm to about 25 μm, about 11 μm to about 13 μm, about 11 μm to about 15 μm, about 11 μm to about 17 μm, about 11 μm to about 19 μm, about 11 μm to about 21 μm, about 11 μm to about 23 μm, about 11 μm to about 25 μm, about 13 μm to about 15 μm, about 13 μm to about 17 μm, about 13 μm to about 19 μm, about 13 μm to about 21 μm, about 13 μm to about 23 μm, about 13 μm to about 25 μm, about 15 μm to about 17 μm, about 15 μm to about 19 μm, about 15 μm to about 21 μm, about 15 μm to about 23 μm, about 15 μm to about 25 μm, about 17 μm to about 19 μm, about 17 μm to about 21 μm, about 17 μm to about 23 μm, about 17 μm to about 25 μm, about 19 μm to about 21 μm, about 19 μm to about 23 μm, about 19 μm to about 25 μm, about 21 μm to about 23 μm, about 21 μm to about 25 μm, or about 23 μm to about 25 μm. In some embodiments the total thickness of the metal plating 310 is about 5 μm, about 7 μm, about 9 μm, about 11 μm, about 13 μm, about 15 μm, about 17 μm, about 19 μm, about 21 μm, about 23 μm, or about 25 μm. In some embodiments, the total thickness of the metal plating 310 is at least about 5 μm, about 7 μm, about 9 μm, about 11 μm, about 13 μm, about 15 μm, about 17 μm, about 19 μm, about 21 μm, or about 23 μm. In some embodiments, the total thickness of the metal plating 310 is at most about 7 μm, about 9 μm, about 11 μm, about 13 μm, about 15 μm, about 17 μm, about 19 μm, about 21 μm, about 23 μm, or about 25 μm.

In some embodiments, each needle of the plurality of needles 212 on the contact surface 211 may further comprise an electrode coating (i.e., electroactive cage 320). In some embodiments, the electroactive cage 320 may be a polymeric coating. In some embodiments, the electroactive cage 320 may comprise a plurality of coating layers. For example, each needle of the plurality of needles 212 may independently be coated with one or more polymeric coating layers forming the electroactive cage 320.

In some embodiments, the total thickness of the electroactive cage 320 on each needle of the plurality of needles 212 is about 5 μm to about 25 μm. In some embodiments the total thickness of the electroactive cage 320 is about 5 μm to about 7 μm, about 5 μm to about 9 μm, about 5 μm to about 11 μm, about 5 μm to about 13 μm, about 5 μm to about 15 μm, about 5 μm to about 17 μm, about 5 μm to about 19 μm, about 5 μm to about 21 μm, about 5 μm to about 23 μm, about 5 μm to about 25 μm, about 7 μm to about 9 μm, about 7 μm to about 11 μm, about 7 μm to about 13 μm, about 7 μm to about 15 μm, about 7 μm to about 17 μm, about 7 μm to about 19 μm, about 7 μm to about 21 μm, about 7 μm to about 23 μm, about 7 μm to about 25 μm, about 9 μm to about 11 μm, about 9 μm to about 13 μm, about 9 μm to about 15 μm, about 9 μm to about 17 μm, about 9 μm to about 19 μm, about 9 μm to about 21 μm, about 9 μm to about 23 μm, about 9 μm to about 25 μm, about 11 μm to about 13 μm, about 11 μm to about 15 μm, about 11 μm to about 17 μm, about 11 μm to about 19 μm, about 11 μm to about 21 μm, about 11 μm to about 23 μm, about 11 μm to about 25 μm, about 13 μm to about 15 μm, about 13 μm to about 17 μm, about 13 μm to about 19 μm, about 13 μm to about 21 μm, about 13 μm to about 23 μm, about 13 μm to about 25 μm, about 15 μm to about 17 μm, about 15 μm to about 19 μm, about 15 μm to about 21 μm, about 15 μm to about 23 μm, about 15 μm to about 25 μm, about 17 μm to about 19 μm, about 17 μm to about 21 μm, about 17 μm to about 23 μm, about 17 μm to about 25 μm, about 19 μm to about 21 μm, about 19 μm to about 23 μm, about 19 μm to about 25 μm, about 21 μm to about 23 μm, about 21 μm to about 25 μm, or about 23 μm to about 25 μm. In some embodiments the total thickness of the electroactive cage 320 is about 5 μm, about 7 μm, about 9 μm, about 11 μm, about 13 μm, about 15 μm, about 17 μm, about 19 μm, about 21 μm, about 23 μm, or about 25 μm. In some embodiments, the total thickness of the electroactive cage 320 is at least about 5 μm, about 7 μm, about 9 μm, about 11 μm, about 13 μm, about 15 μm, about 17 μm, about 19 μm, about 21 μm, or about 23 μm. In some embodiments, the total thickness of the electroactive cage 320 is at most about 7 μm, about 9 μm, about 11 μm, about 13 μm, about 15 μm, about 17 μm, about 19 μm, about 21 μm, about 23 μm, or about 25 μm.

In some embodiments, the electroactive cage 320 may comprise both electroactive composite and a stabilizing polymer matrix. In some embodiments, the electroactive composite and the stabilizing polymer matrix may self-assemble to form the electroactive cage 320.

In some embodiments, the electroactive composite comprises a polymer selected from polyethyleneimine (PEI), polyethylene (PE), polydimethyl siloxane (PDMS), polytetrafluoroethylene (PTFE), or combinations thereof.

In some embodiments, the stabilizing polymer matrix may comprise an alkyl ammonium salt and a sulfonated tetrafluoroethylene polymer.

In some embodiments, the electroactive cage 320 comprises a polymer with an average weight of about 5 kD to about 1,000 kD. In some embodiments, the electroactive cage 320 comprises a polymer with an average weight of about 5 kD to about 10 kD, about 5 kD to about 15 kD, about 5 kD to about 20 kD, about 5 kD to about 25 kD, about 5 kD to about 30 kD, about 5 kD to about 50 kD, about 5 kD to about 100 kD, about 5 kD to about 200 kD, about 5 kD to about 500 kD, about 5 kD to about 1,000 kD, about 10 kD to about 15 kD, about 10 kD to about 20 kD, about 10 kD to about 25 kD, about 10 kD to about 30 kD, about 10 kD to about 50 kD, about 10 kD to about 100 kD, about 10 kD to about 200 kD, about 10 kD to about 500 kD, about 10 kD to about 1,000 kD, about 15 kD to about 20 kD, about 15 kD to about 25 kD, about 15 kD to about 30 kD, about 15 kD to about 50 kD, about 15 kD to about 100 kD, about 15 kD to about 200 kD, about 15 kD to about 500 kD, about 15 kD to about 1,000 kD, about 20 kD to about 25 kD, about 20 kD to about 30 kD, about 20 kD to about 50 kD, about 20 kD to about 100 kD, about 20 kD to about 200 kD, about 20 kD to about 500 kD, about 20 kD to about 1,000 kD, about 25 kD to about 30 kD, about 25 kD to about 50 kD, about 25 kD to about 100 kD, about 25 kD to about 200 kD, about 25 kD to about 500 kD, about 25 kD to about 1,000 kD, about 30 kD to about 50 kD, about 30 kD to about 100 kD, about 30 kD to about 200 kD, about 30 kD to about 500 kD, about 30 kD to about 1,000 kD, about 50 kD to about 100 kD, about 50 kD to about 200 kD, about 50 kD to about 500 kD, about 50 kD to about 1,000 kD, about 100 kD to about 200 kD, about 100 kD to about 500 kD, about 100 kD to about 1,000 kD, about 200 kD to about 500 kD, about 200 kD to about 1,000 kD, or about 500 kD to about 1,000 kD. In some embodiments, the electroactive cage 320 comprises a polymer with an average weight of about 5 kD, about 10 kD, about 15 kD, about 20 kD, about 25 kD, about 30 kD, about 50 kD, about 100 kD, about 200 kD, about 500 kD, or about 1,000 kD. In some embodiments, the electroactive cage 320 comprises a polymer with an average weight of about 5 kD, about 10 kD, about 15 kD, about 20 kD, about 25 kD, about 30 kD, about 50 kD, about 100 kD, about 200 kD, or about 500 kD. In some embodiments, the electroactive cage 320 comprises a polymer with an average weight of at most about 10 kD, about 15 kD, about 20 kD, about 25 kD, about 30 kD, about 50 kD, about 100 kD, about 200 kD, about 500 kD, or about 1,000 kD.

In some embodiments, the electroactive cage 320 comprises a polymer with a polydispersity of about 1 to about 5. In some embodiments, the electroactive cage 320 comprises a polymer with a polydispersity of about 1 to about 1.2, about 1 to about 1.5, about 1 to about 1.7, about 1 to about 2, about 1 to about 2.2, about 1 to about 2.5, about 1 to about 3, about 1 to about 3.5, about 1 to about 4, about 1 to about 4.5, about 1 to about 5, about 1.2 to about 1.5, about 1.2 to about 1.7, about 1.2 to about 2, about 1.2 to about 2.2, about 1.2 to about 2.5, about 1.2 to about 3, about 1.2 to about 3.5, about 1.2 to about 4, about 1.2 to about 4.5, about 1.2 to about 5, about 1.5 to about 1.7, about 1.5 to about 2, about 1.5 to about 2.2, about 1.5 to about 2.5, about 1.5 to about 3, about 1.5 to about 3.5, about 1.5 to about 4, about 1.5 to about 4.5, about 1.5 to about 5, about 1.7 to about 2, about 1.7 to about 2.2, about 1.7 to about 2.5, about 1.7 to about 3, about 1.7 to about 3.5, about 1.7 to about 4, about 1.7 to about 4.5, about 1.7 to about 5, about 2 to about 2.2, about 2 to about 2.5, about 2 to about 3, about 2 to about 3.5, about 2 to about 4, about 2 to about 4.5, about 2 to about 5, about 2.2 to about 2.5, about 2.2 to about 3, about 2.2 to about 3.5, about 2.2 to about 4, about 2.2 to about 4.5, about 2.2 to about 5, about 2.5 to about 3, about 2.5 to about 3.5, about 2.5 to about 4, about 2.5 to about 4.5, about 2.5 to about 5, about 3 to about 3.5, about 3 to about 4, about 3 to about 4.5, about 3 to about 5, about 3.5 to about 4, about 3.5 to about 4.5, about 3.5 to about 5, about 4 to about 4.5, about 4 to about 5, or about 4.5 to about 5. In some embodiments, the electroactive cage 320 comprises a polymer with a polydispersity of about 1, about 1.2, about 1.5, about 1.7, about 2, about 2.2, about 2.5, about 3, about 3.5, about 4, about 4.5, or about 5. In some embodiments, the electroactive cage 320 comprises a polymer with a polydispersity of about 1, about 1.2, about 1.5, about 1.7, about 2, about 2.2, about 2.5, about 3, about 3.5, about 4, or about 4.5. In some embodiments, the electroactive cage 320 comprises a polymer with a polydispersity of at most about 1.2, about 1.5, about 1.7, about 2, about 2.2, about 2.5, about 3, about 3.5, about 4, about 4.5, or about 5.

In some embodiments, the electroactive composite further comprises a polymer and multiwalled carbon nanotubes (MWCNT). In some embodiments, the MWCNT may be disposed on or embedded in the polymer of the electroactive composite.

In some embodiments, the average internal diameter of the MWCNT is about 0.5 nm to about 100 nm. In some embodiments, the average internal diameter of the MWCNT is about 0.5 nm to about 2 nm, about 0.5 nm to about 3 nm, about 0.5 nm to about 5 nm, about 0.5 nm to about 7 nm, about 0.5 nm to about 10 nm, about 0.5 nm to about 15 nm, about 0.5 nm to about 20 nm, about 0.5 nm to about 25 nm, about 0.5 nm to about 30 nm, about 0.5 nm to about 35 nm, about 0.5 nm to about 40 nm, about 0.5 nm to about 100 nm, about 2 nm to about 3 nm, about 2 nm to about 5 nm, about 2 nm to about 7 nm, about 2 nm to about 10 nm, about 2 nm to about 15 nm, about 2 nm to about 20 nm, about 2 nm to about 25 nm, about 2 nm to about 30 nm, about 2 nm to about 35 nm, about 2 nm to about 40 nm, about 2 nm to about 100 nm, about 3 nm to about 5 nm, about 3 nm to about 7 nm, about 3 nm to about 10 nm, about 3 nm to about 15 nm, about 3 nm to about 20 nm, about 3 nm to about 25 nm, about 3 nm to about 30 nm, about 3 nm to about 35 nm, about 3 nm to about 40 nm, about 3 nm to about 100 nm, about 5 nm to about 7 nm, about 5 nm to about 10 nm, about 5 nm to about 15 nm, about 5 nm to about 20 nm, about 5 nm to about 25 nm, about 5 nm to about 30 nm, about 5 nm to about 35 nm, about 5 nm to about 40 nm, about 5 nm to about 100 nm, about 7 nm to about 10 nm, about 7 nm to about 15 nm, about 7 nm to about 20 nm, about 7 nm to about 25 nm, about 7 nm to about 30 nm, about 7 nm to about 35 nm, about 7 nm to about 40 nm, about 7 nm to about 100 nm, about 10 nm to about 15 nm, about 10 nm to about 20 nm, about 10 nm to about 25 nm, about 10 nm to about 30 nm, about 10 nm to about 35 nm, about 10 nm to about 40 nm, about 10 nm to about 100 nm, about 15 nm to about 20 nm, about 15 nm to about 25 nm, about 15 nm to about 30 nm, about 15 nm to about 35 nm, about 15 nm to about 40 nm, about 15 nm to about 100 nm, about 20 nm to about 25 nm, about 20 nm to about 30 nm, about 20 nm to about 35 nm, about 20 nm to about 40 nm, about 20 nm to about 100 nm, about 25 nm to about 30 nm, about 25 nm to about 35 nm, about 25 nm to about 40 nm, about 25 nm to about 100 nm, about 30 nm to about 35 nm, about 30 nm to about 40 nm, about 30 nm to about 100 nm, about 35 nm to about 40 nm, about 35 nm to about 100 nm, or about 40 nm to about 100 nm. In some embodiments, the average internal diameter of the MWCNT is about 0.5 nm, about 2 nm, about 3 nm, about 5 nm, about 7 nm, about 10 nm, about 15 nm, about 20 nm, about 25 nm, about 30 nm, about 35 nm, about 40 nm, or about 100 nm. In some embodiments, the average internal diameter of the MWCNT is at least about 0.5 nm, about 2 nm, about 3 nm, about 5 nm, about 7 nm, about 10 nm, about 15 nm, about 20 nm, about 25 nm, about 30 nm, about 35 nm, or about 40 nm. In some embodiments, the average internal diameter of the MWCNT is at most about 2 nm, about 3 nm, about 5 nm, about 7 nm, about 10 nm, about 15 nm, about 20 nm, about 25 nm, about 30 nm, about 35 nm, about 40 nm, or about 100 nm.

In some embodiments, the average length of the MWCNT is about 5 μm to about 100 μm. In some embodiments, the average length of the MWCNT is about 5 μm to about 7 μm, about 5 μm to about 10 μm, about 5 μm to about 12 μm, about 5 μm to about 15 μm, about 5 μm to about 20 μm, about 5 μm to about 25 μm, about 5 μm to about 30 μm, about 5 μm to about 40 μm, about 5 μm to about 50 μm, about 5 μm to about 75 μm, about 5 μm to about 100 μm, about 7 μm to about 10 μm, about 7 μm to about 12 μm, about 7 μm to about 15 μm, about 7 μm to about 20 μm, about 7 μm to about 25 μm, about 7 μm to about 30 μm, about 7 μm to about 40 μm, about 7 μm to about 50 μm, about 7 μm to about 75 μm, about 7 μm to about 100 μm, about 10 μm to about 12 μm, about 10 μm to about 15 μm, about 10 μm to about 20 μm, about 10 μm to about 25 μm, about 10 μm to about 30 μm, about 10 μm to about 40 μm, about 10 μm to about 50 μm, about 10 μm to about 75 μm, about 10 μm to about 100 μm, about 12 μm to about 15 μm, about 12 μm to about 20 μm, about 12 μm to about 25 μm, about 12 μm to about 30 μm, about 12 μm to about 40 μm, about 12 μm to about 50 μm, about 12 μm to about 75 μm, about 12 μm to about 100 μm, about 15 μm to about 20 μm, about 15 μm to about 25 μm, about 15 μm to about 30 μm, about 15 μm to about 40 μm, about 15 μm to about 50 μm, about 15 μm to about 75 μm, about 15 μm to about 100 μm, about 20 μm to about 25 μm, about 20 μm to about 30 μm, about 20 μm to about 40 μm, about 20 μm to about 50 μm, about 20 μm to about 75 μm, about 20 μm to about 100 μm, about 25 μm to about 30 μm, about 25 μm to about 40 μm, about 25 μm to about 50 μm, about 25 μm to about 75 μm, about 25 μm to about 100 μm, about 30 μm to about 40 μm, about 30 μm to about 50 μm, about 30 μm to about 75 μm, about 30 μm to about 100 μm, about 40 μm to about 50 μm, about 40 μm to about 75 μm, about 40 μm to about 100 μm, about 50 μm to about 75 μm, about 50 μm to about 100 μm, or about 75 μm to about 100 μm. In some embodiments, the average length of the MWCNT is about 5 μm, about 7 μm, about 10 μm, about 12 μm, about 15 μm, about 20 μm, about 25 μm, about 30 μm, about 40 μm, about 50 μm, about 75 μm, or about 100 μm. In some embodiments, the average length of the MWCNT is at least about 5 μm, about 7 μm, about 10 μm, about 12 μm, about 15 μm, about 20 μm, about 25 μm, about 30 μm, about 40 μm, about 50 μm, or about 75 μm. In some embodiments, the average length of the MWCNT is at most about 7 μm, about 10 μm, about 12 μm, about 15 μm, about 20 μm, about 25 μm, about 30 μm, about 40 μm, about 50 μm, about 75 μm, or about 100 μm.

In some embodiments, each needle of the plurality of needles 212 on the contact surface 211 may further comprise a hydrogel coating 330. In some embodiments, all needles of the plurality of needles may include a hydrogel coating 330. In some embodiments, the hydrogel coating 330 is configured to protect the electroactive cage 320 from the deposition of molecular redox byproducts.

In some embodiments, the hydrogel 330 coating may be an agarose-based hydrogel. In some embodiments, the hydrogel coating 330 may be doped with an electrolyte or a solution containing an electrolyte in order to increase the electrical conductivity of the hydrogel. In some embodiments, the electrolyte may be a metal chloride, a metal sulfate, a metal nitrate, a metal nitrite, or combinations thereof. In some embodiments, the electrolyte may be LiCl, NaCl, KCl, CaCl₂, MgCl₂, or combinations thereof. In some embodiments, the electrolyte may be an alkyl-ammonium salt.

In some embodiments the total thickness of the hydrogel coating 330 on each needle of the plurality of needles 212 is about 5 μm to about 25 μm. In some embodiments the total thickness of the hydrogel coating 330 is about 5 μm to about 7 μm, about 5 μm to about 9 μm, about 5 μm to about 11 μm, about 5 μm to about 13 μm, about 5 μm to about 15 μm, about 5 μm to about 17 μm, about 5 μm to about 19 μm, about 5 μm to about 21 μm, about 5 μm to about 23 μm, about 5 μm to about 25 μm, about 7 μm to about 9 μm, about 7 μm to about 11 μm, about 7 μm to about 13 μm, about 7 μm to about 15 μm, about 7 μm to about 17 μm, about 7 μm to about 19 μm, about 7 μm to about 21 μm, about 7 μm to about 23 μm, about 7 μm to about 25 μm, about 9 μm to about 11 μm, about 9 μm to about 13 μm, about 9 μm to about 15 μm, about 9 μm to about 17 μm, about 9 μm to about 19 μm, about 9 μm to about 21 μm, about 9 μm to about 23 μm, about 9 μm to about 25 μm, about 11 μm to about 13 μm, about 11 μm to about 15 μm, about 11 μm to about 17 μm, about 11 μm to about 19 μm, about 11 μm to about 21 μm, about 11 μm to about 23 μm, about 11 μm to about 25 μm, about 13 μm to about 15 μm, about 13 μm to about 17 μm, about 13 μm to about 19 μm, about 13 μm to about 21 μm, about 13 μm to about 23 μm, about 13 μm to about 25 μm, about 15 μm to about 17 μm, about 15 μm to about 19 μm, about 15 μm to about 21 μm, about 15 μm to about 23 μm, about 15 μm to about 25 μm, about 17 μm to about 19 μm, about 17 μm to about 21 μm, about 17 μm to about 23 μm, about 17 μm to about 25 μm, about 19 μm to about 21 μm, about 19 μm to about 23 μm, about 19 μm to about 25 μm, about 21 μm to about 23 μm, about 21 μm to about 25 μm, or about 23 μm to about 25 μm. In some embodiments the total thickness of the hydrogel coating 330 is about 5 μm, about 7 μm, about 9 μm, about 11 μm, about 13 μm, about 15 μm, about 17 μm, about 19 μm, about 21 μm, about 23 μm, or about 25 μm. In some embodiments, the total thickness of the hydrogel coating 330 is at least about 5 μm, about 7 μm, about 9 μm, about 11 μm, about 13 μm, about 15 μm, about 17 μm, about 19 μm, about 21 μm, or about 23 μm. In some embodiments, the total thickness of the hydrogel coating 330 is at most about 7 μm, about 9 μm, about 11 μm, about 13 μm, about 15 μm, about 17 μm, about 19 μm, about 21 μm, about 23 μm, or about 25 μm.

In some embodiments, the microneedle array 220 may further comprise a plurality of electrically isolated domains. FIG. 3, illustrates an embodiment with three electrically isolated domains. In some embodiments, the plurality of electrically isolated domains may be a plurality of electrodes. In some embodiments, the plurality of electrodes may each independently be selected from a working electrode 301, a reference electrode 302, and a counter electrode 303. In some embodiments, the plurality of electrically isolated domains may be two domains comprising a working electrode 301 and a combined reference and counter electrode. In some embodiments, at least one of the working electrode 301, the reference electrode 302, and the counter electrode 303 comprises gold, carbon, platinum, or combinations thereof.

In some embodiments, at least one of the working electrode 301, the reference electrode 302, and the counter electrode 303, is coated with an electrode coating. In some embodiments, the electrode coating may comprise a quaternary ammonium salt, a sulfonated tetrafluoroethylene polymer, a polyethyleneimine polymer functionalized with MWCNT, or combinations thereof. In some embodiments, the electrode coating improves conductivity for more accurate and/or precise measurement, increases the strength and/or durability of the electrodes, or both.

In some embodiments, each needle of the plurality of needles 212 on the microneedle array 210 may serve as either the working electrode 301, the reference electrode 302, or the counter electrode 303 for the electrochemical sensing process, enabling direct detection of analytes inside the human body.

In some embodiments, each needle of the plurality of needles 212 serving as the working electrode 301 may comprise gold or platinum metal plating 310 and an electroactive cage 320. In some embodiments, the electroactive cage 320 may comprise a quaternary ammonium salt, a sulfonated tetrafluoroethylene polymer, polyethyleneimine polymer functionalized with MWCNT, or combinations thereof.

In some embodiments, each needle of the plurality of needles 212 serving as the reference electrode 302 may comprise silver, silver chloride, or combinations thereof. In some embodiments, each needle of the plurality of needles 212 serving as the counter electrode 303 may comprise gold, platinum, carbon, or combinations thereof.

In some embodiments, each need of the plurality of needles 212 serving as either the working electrode 301, the reference electrode 302, the counter electrode 303, or combinations thereof, may be coated with a hydrogel coating 330.

When the plurality of needles 212 on the contact surface 211 of the microneedle array 210 are non-uniformly spaced, the pitch of the array may either describe the distance between a needle of the plurality of needles 212 on the working electrode 301 and a needle on the counter electrode 303; between a needle of the plurality of needles 212 on the working electrode 301 and a needle on the reference electrode 302, or between a needle of the plurality of needles 212 on the reference electrode 302 and a needle on the counter electrode 303.

In some embodiments, the microneedle array 210 of the sensor 200 is operably coupled to the body 220. In some embodiments, the body 220 of the sensor 200 is configured to be attached to be attached to the skin of a subject. In some embodiments, the body 220 is attached to a subject's skin using an adhesive. In some embodiments, each needle of the microneedle array 212 on the contact surface 211 of the microneedle array 210, may penetrate the skin of a subject when the sensor 200 is attached.

In some embodiments, the sensor 220 is configured to be attached to the skin of a subject for about 1 day to about 30 days.

In some embodiments, the sensor 220 is configured to be attached to the skin of a subject for at least about 30 days without substantial biofouling (i.e., substantial loss of the functionality or accuracy of the sensor).

In some embodiments, the sensor 200 comprises a COM module 230 operably coupled to the microneedle array 210. The COM module 230 may comprise a potentiostat, a processor coupled to the potentiostat, and a memory coupled to the processor.

In some embodiments, the potentiostat of the COM module 230 provides a sweeping voltage potential between the working electrode 301 and the reference electrode 302 of the microneedle array 210. In some embodiments, the potentiostat further measures a current flowing between the working electrode 301 and the counter electrode 303.

In some embodiments, the processor of the COM module 230 may be configured to drive and/or control the electrochemical measurements performed using the potentiostat. In some embodiments, the processor analyzes data from the potentiostat.

The memory of the COM module 230 may comprise instructions for the processor to apply a sweeping voltage to the potentiostat, measure a current in response to applying the sweeping voltage, and determine using the measured current the presence, absence, or concentration of an analyte.

In some embodiments, the COM module 230 stores sensor data onto the memory on a temporary basis. In some embodiments, the COM module 230 may further comprise a receiver and/or a transmitter that is coupled to the processor and is configured to transfer data between the sensor 200 to a to one or more of an external device, system, or network. In some embodiments, the transmitter is configured to transmit a signal based on the presence or absence of at least one analyte, the measured current, or both, to one or more of an external device, system, or network. In some embodiments, the signal transmitted by the transmitter is based on a measured concentration of the at least one analyte.

In some embodiments, the memory further comprises instructions to transmit the signal periodically at about 1 min to about 6-hour intervals. In some embodiments, the memory comprises instructions to transmit the signal over a period of about 5 seconds to about 30 second, to about 60 seconds, to about 2 minutes, to about 5 minutes, to about 10 minutes, to about 20 minutes, to about 30 minutes, to about 45 minutes, to about 60 minutes, to about 2 hours, to about 3 hours, to about 4 hours, to about 5 hours, or to about 6 hours. the memory comprises instructions to transmit the signal over a period of no greater than about 6 hours, about 5 hours, about 4 hours, about 3 hours, about 2 hours, about 1 hour, about 45 minutes, about 30 minutes, about 20 minutes, about 10 minutes, about 5 minutes, about 2 minutes, about 1 minute, about 30 seconds.

### Computing System

In some embodiments, the sensor 200 is operably coupled to a computer system 400.

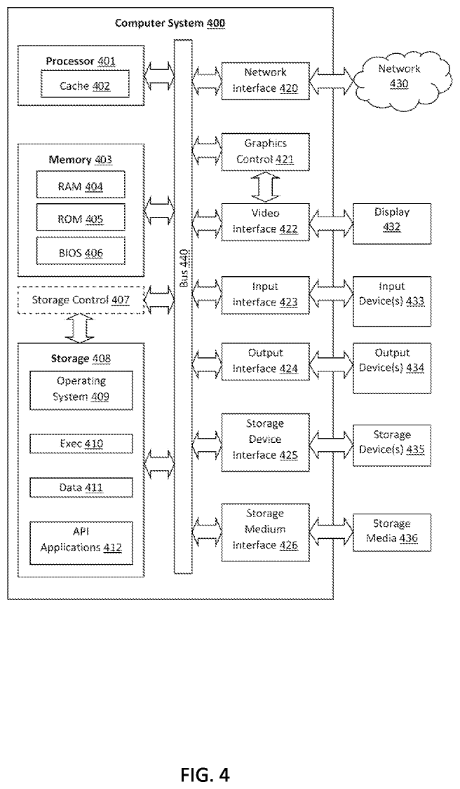

Referring to FIG. 4, a block diagram is shown depicting an exemplary machine that includes a computer system 400 (e.g., a processing or computing system) within which a set of instructions can execute for causing a device to perform or execute any one or more of the aspects and/or methodologies for static code scheduling of the present disclosure. The components in FIG. 4 are examples only and do not limit the scope of use or functionality of any hardware, software, embedded logic component, or a combination of two or more such components implementing particular embodiments.

Computer system 400 may include one or more processors 401, a memory 403, and a storage 408 that communicate with each other, and with other components, via a bus 440. The bus 440 may also link a display 432, one or more input devices 433 (which may, for example, include a keypad, a keyboard, a mouse, a stylus, etc.), one or more output devices 434, one or more storage devices 435, and various tangible storage media 436. All elements may interface directly or via one or more interfaces or adaptors to the bus 440. For instance, the various tangible storage media 436 can interface with the bus 440 via storage medium interface 426. Computer system 400 may have any suitable physical form, including but not limited to one or more integrated circuits (ICs), printed circuit boards (PCBs), mobile handheld devices (such as mobile telephones or PDAs), laptop or notebook computers, distributed computer systems, computing grids, or servers.

Computer system 400 includes one or more processor(s) 401 (e.g., central processing units (CPUs) or general purpose graphics processing units (GPGPUs)) that carry out functions. Processor(s) 401 optionally contains a cache memory unit 402 for temporary local storage of instructions, data, or computer addresses. Processor(s) 401 are configured to assist in execution of computer readable instructions. Computer system 400 may provide functionality for the components depicted in FIG. 4 as a result of the processor(s) 401 executing non-transitory, processor-executable instructions embodied in one or more tangible computer-readable storage media, such as memory 403, storage 408, storage devices 435, and/or storage medium 436. The computer-readable media may store software that implements particular embodiments, and processor(s) 401 may execute the software. Memory 403 may read the software from one or more other computer-readable media (such as mass storage device(s) 435, 436) or from one or more other sources through a suitable interface, such as network interface 420. The software may cause processor(s) 401 to carry out one or more processes or one or more steps of one or more processes described or illustrated herein. Carrying out such processes or steps may include defining data structures stored in memory 403 and modifying the data structures as directed by the software.

The memory 403 may include various components (e.g., machine readable media) including, but not limited to, a random access memory component (e.g., RAM 404) (e.g., static RAM (SRAM), dynamic RAM (DRAM), ferroelectric random access memory (FRAM), phase-change random access memory (PRAM), etc.), a read-only memory component (e.g., ROM 405), and any combinations thereof. ROM 405 may act to communicate data and instructions unidirectionally to processor(s) 401, and RAM 404 may act to communicate data and instructions bidirectionally with processor(s) 401. ROM 405 and RAM 404 may include any suitable tangible computer-readable media described below. In one example, a basic input/output system 406 (BIOS), including basic routines that help to transfer information between elements within computer system 400, such as during start-up, may be stored in the memory 403.

Fixed storage 408 is connected bidirectionally to processor(s) 401, optionally through storage control unit 407. Fixed storage 408 provides additional data storage capacity and may also include any suitable tangible computer-readable media described herein. Storage 408 may be used to store operating system 409, executable(s) 410, data 411, applications 412 (application programs), and the like. Storage 408 can also include an optical disk drive, a solid-state memory device (e.g., flash-based systems), or a combination of any of the above. Information in storage 408 may, in appropriate cases, be incorporated as virtual memory in memory 403.

In one example, storage device(s) 435 may be removably interfaced with computer system 400 (e.g., via an external port connector (not shown)) via a storage device interface 425. Particularly, storage device(s) 435 and an associated machine-readable medium may provide non-volatile and/or volatile storage of machine-readable instructions, data structures, program modules, and/or other data for the computer system 400. In one example, software may reside, completely or partially, within a machine-readable medium on storage device(s) 435. In another example, software may reside, completely or partially, within processor(s) 401.

Bus 440 connects a wide variety of subsystems. Herein, reference to a bus may encompass one or more digital signal lines serving a common function, where appropriate. Bus 440 may be any of several types of bus structures including, but not limited to, a memory bus, a memory controller, a peripheral bus, a local bus, and any combinations thereof, using any of a variety of bus architectures. As an example, and not by way of limitation, such architectures include an Industry Standard Architecture (ISA) bus, an Enhanced ISA (EISA) bus, a Micro Channel Architecture (MCA) bus, a Video Electronics Standards Association local bus (VLB), a Peripheral Component Interconnect (PCI) bus, a PCI-Express (PCI-X) bus, an Accelerated Graphics Port (AGP) bus, HyperTransport (HTX) bus, serial advanced technology attachment (SATA) bus, and any combinations thereof.

Computer system 400 may also include an input device 433. In one example, a user of computer system 400 may enter commands and/or other information into computer system 400 via input device(s) 433. Examples of an input device(s) 433 include, but are not limited to, an alpha-numeric input device (e.g., a keyboard), a pointing device (e.g., a mouse or touchpad), a touchpad, a touch screen, a multi-touch screen, a joystick, a stylus, a gamepad, an audio input device (e.g., a microphone, a voice response system, etc.), an optical scanner, a video or still image capture device (e.g., a camera), and any combinations thereof. In some embodiments, the input device is a Kinect, Leap Motion, or the like. Input device(s) 433 may be interfaced to bus 440 via any of a variety of input interfaces 423 (e.g., input interface 423) including, but not limited to, serial, parallel, game port, USB, FIREWIRE, THUNDERBOLT, or any combination of the above.

In particular embodiments, when computer system 400 is connected to network 430, computer system 400 may communicate with other devices, specifically mobile devices and enterprise systems, distributed computing systems, cloud storage systems, cloud computing systems, and the like, connected to network 430. Communications to and from computer system 400 may be sent through network interface 420. For example, network interface 420 may receive incoming communications (such as requests or responses from other devices) in the form of one or more packets (such as Internet Protocol (IP) packets) from network 430, and computer system 400 may store the incoming communications in memory 403 for processing. Computer system 400 may similarly store outgoing communications (such as requests or responses to other devices) in the form of one or more packets in memory 403 and communicated to network 430 from network interface 420. Processor(s) 401 may access these communication packets stored in memory 403 for processing.

Examples of the network interface 420 include, but are not limited to, a network interface card, a modem, and any combination thereof. Examples of a network 430 or network segment 430 include, but are not limited to, a distributed computing system, a cloud computing system, a wide area network (WAN) (e.g., the Internet, an enterprise network), a local area network (LAN) (e.g., a network associated with an office, a building, a campus or other relatively small geographic space), a telephone network, a direct connection between two computing devices, a peer-to-peer network, and any combinations thereof. A network, such as network 430, may employ a wired and/or a wireless mode of communication. In general, any network topology may be used.

Information and data can be displayed through a display 432. Examples of a display 432 include, but are not limited to, a cathode ray tube (CRT), a liquid crystal display (LCD), a thin film transistor liquid crystal display (TFT-LCD), an organic liquid crystal display (OLED) such as a passive-matrix OLED (PMOLED) or active-matrix OLED (AMOLED) display, a plasma display, and any combinations thereof. The display 432 can interface to the processor(s) 401, memory 403, and fixed storage 408, as well as other devices, such as input device(s) 433, via the bus 440. The display 432 is linked to the bus 440 via a video interface 422, and transport of data between the display 432 and the bus 440 can be controlled via the graphics control 421. In some embodiments, the display is a video projector. In some embodiments, the display is a head-mounted display (HMD) such as a VR headset. In further embodiments, suitable VR headsets include, by way of non-limiting examples, HTC Vive, Oculus Rift, Samsung Gear VR, Microsoft HoloLens, Razer OSVR, FOVE VR, Zeiss VR One, Avegant Glyph, Freefly VR headset, and the like. In still further embodiments, the display is a combination of devices such as those disclosed herein.

In addition to a display 432, computer system 400 may include one or more other peripheral output devices 434 including, but not limited to, an audio speaker, a printer, a storage device, and any combinations thereof. Such peripheral output devices may be connected to the bus 440 via an output interface 424. Examples of an output interface 424 include, but are not limited to, a serial port, a parallel connection, a USB port, a FIREWIRE port, a THUNDERBOLT port, and any combinations thereof.

In addition, or as an alternative, computer system 400 may provide functionality as a result of logic hardwired or otherwise embodied in a circuit, which may operate in place of or together with software to execute one or more processes or one or more steps of one or more processes described or illustrated herein. Reference to software in this disclosure may encompass logic, and reference to logic may encompass software. Moreover, reference to a computer-readable medium may encompass a circuit (such as an IC) storing software for execution, a circuit embodying logic for execution, or both, where appropriate. The present disclosure encompasses any suitable combination of hardware, software, or both.

Those of skill in the art will appreciate that the various illustrative logical blocks, modules, circuits, and algorithm steps described in connection with the embodiments disclosed herein may be implemented as electronic hardware, computer software, or combinations of both. To clearly illustrate this interchangeability of hardware and software, various illustrative components, blocks, modules, circuits, and steps have been described above generally in terms of their functionality.

The various illustrative logical blocks, modules, and circuits described in connection with the embodiments disclosed herein may be implemented or performed with a general purpose processor, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A general purpose processor may be a microprocessor, but in the alternative, the processor may be any conventional processor, controller, microcontroller, or state machine. A processor may also be implemented as a combination of computing devices, e.g., a combination of a DSP and a microprocessor, a plurality of microprocessors, one or more microprocessors in conjunction with a DSP core, or any other such configuration.

The steps of a method or algorithm described in connection with the embodiments disclosed herein may be embodied directly in hardware, in a software module executed by one or more processor(s), or in a combination of the two. A software module may reside in RAM memory, flash memory, ROM memory, EPROM memory, EEPROM memory, registers, hard disk, a removable disk, a CD-ROM, or any other form of storage medium known in the art. An exemplary storage medium is coupled to the processor such the processor can read information from, and write information to, the storage medium. In the alternative, the storage medium may be integral to the processor. The processor and the storage medium may reside in an ASIC. The ASIC may reside in a user terminal. In the alternative, the processor and the storage medium may reside as discrete components in a user terminal.

In accordance with the description herein, suitable computing devices include, by way of non-limiting examples, server computers, desktop computers, laptop computers, notebook computers, sub-notebook computers, netbook computers, netpad computers, set-top computers, media streaming devices, handheld computers, Internet appliances, mobile smartphones, tablet computers, personal digital assistants, video game consoles, and vehicles. Those of skill in the art will also recognize that select televisions, video players, and digital music players with optional computer network connectivity are suitable for use in the system described herein. Suitable tablet computers, in various embodiments, include those with booklet, slate, and convertible configurations, known to those of skill in the art.

In some embodiments, the computing device includes an operating system configured to perform executable instructions. The operating system is, for example, software, including programs and data, which manages the device's hardware and provides services for execution of applications. Those of skill in the art will recognize that suitable server operating systems include, by way of non-limiting examples, FreeBSD, OpenBSD, NetB SD®, Linux, Apple® Mac OS X Server®, Oracle® Solaris®, Windows Server®, and Novell® NetWare®. Those of skill in the art will recognize that suitable personal computer operating systems include, by way of non-limiting examples, Microsoft® Windows®, Apple® Mac OS X®, UNIX®, and UNIX-like operating systems such as GNU/Linux®. In some embodiments, the operating system is provided by cloud computing. Those of skill in the art will also recognize that suitable mobile smartphone operating systems include, by way of non-limiting examples, Nokia® Symbian® OS, Apple® iOS®, Research In Motion® BlackBerry OS®, Google® Android®, Microsoft® Windows Phone® OS, Microsoft® Windows Mobile® OS, Linux®, and Palm® WebOS®. Those of skill in the art will also recognize that suitable media streaming device operating systems include, by way of non-limiting examples, Apple TV®, Roku®, Boxee®, Google TV®, Google Chromecast®, Amazon Fire®, and Samsung® HomeSync®. Those of skill in the art will also recognize that suitable video game console operating systems include, by way of non-limiting examples, Sony® P53®, Sony® P54®, Microsoft® Xbox 360®, Microsoft Xbox One, Nintendo® Wii®, Nintendo® Wii U®, and Ouya®.

In some embodiments, the platforms, systems, media, and methods disclosed herein include one or more non-transitory computer readable storage media encoded with a program including instructions executable by the operating system of an optionally networked computing device. In further embodiments, a computer readable storage medium is a tangible component of a computing device. In still further embodiments, a computer readable storage medium is optionally removable from a computing device. In some embodiments, a computer readable storage medium includes, by way of non-limiting examples, CD-ROMs, DVDs, flash memory devices, solid state memory, magnetic disk drives, magnetic tape drives, optical disk drives, distributed computing systems including cloud computing systems and services, and the like. In some cases, the program and instructions are permanently, substantially permanently, semi-permanently, or non-transitorily encoded on the media.

In some embodiments, the platforms, systems, media, and methods disclosed herein include at least one computer program, or use of the same. A computer program includes a sequence of instructions, executable by one or more processor(s) of the computing device's CPU, written to perform a specified task. Computer readable instructions may be implemented as program modules, such as functions, objects, Application Programming Interfaces (APIs), computing data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft®.NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™ and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

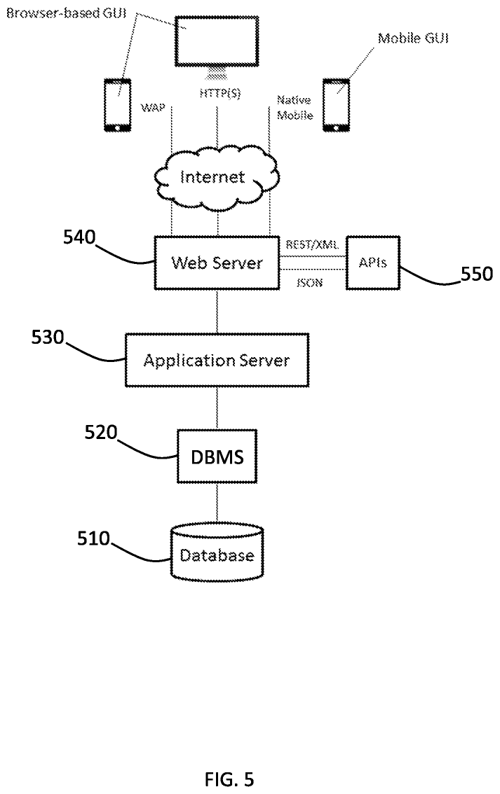

Referring to FIG. 5, in a particular embodiment, an application provision system comprises one or more databases 510 accessed by a database management system (DBMS). In some embodiments, the DBMS may be a relational database management system (RDBMS) 520. Suitable RDBMSs include Firebird, MySQL, PostgreSQL, SQLite, Oracle Database, Microsoft SQL Server, IBM DB2, IBM Informix, SAP Sybase, SAP Sybase, Teradata, and the like. In this embodiment, the application provision system further comprises one or more application severs 530 (such as Java servers, .NET servers, PHP servers, and the like) and one or more web servers 540 (such as Apache, IIS, GWS and the like). The web server(s) optionally expose one or more web services via app application programming interfaces (APIs) 550. Via a network, such as the Internet, the system provides browser-based and/or mobile native user interfaces.

### Cloud Network Architecture

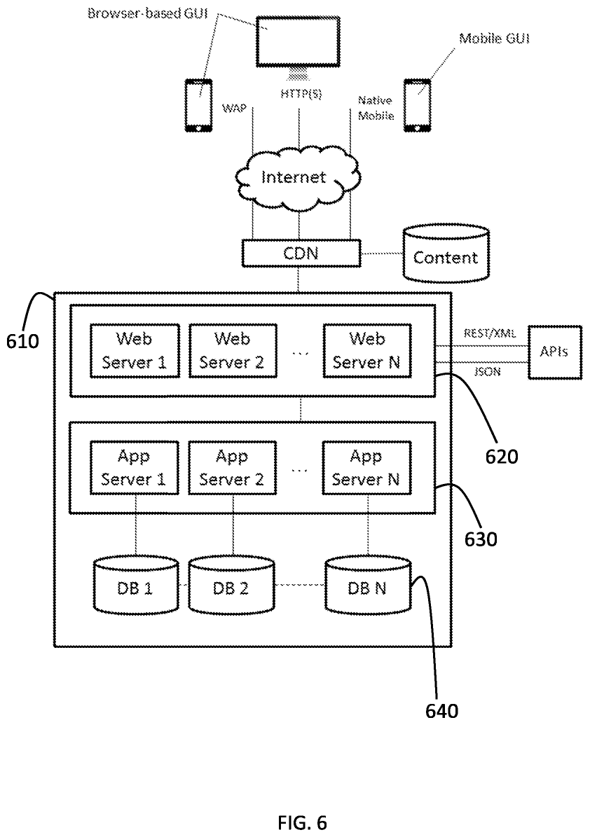

Referring to FIG. 6, in a particular embodiment, an application provision system alternatively has a distributed, cloud-based architecture 610 and comprises elastically load balanced, auto-scaling web server resources 620 and application server resources 630 as well synchronously replicated databases 640.

In some embodiments, a computer program includes a mobile application provided to a mobile computing device. In some embodiments, the mobile application is provided to a mobile computing device at the time it is manufactured. In other embodiments, the mobile application is provided to a mobile computing device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome Web Store, BlackBerry® App World, App Store for Palm devices, App Catalog for webOS, Windows® Marketplace for Mobile, Ovi Store for Nokia® devices, Samsung® Apps, and Nintendo® DSi Shop.

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB.NET, or combinations thereof. Compilation is often performed, at least in part, to create an executable program. In some embodiments, a computer program includes one or more executable complied applications.

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™, PHP, Python™, and VB.NET, or combinations thereof.

Web browsers (also called Internet browsers) are software applications, designed for use with network-connected computing devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called microbrowsers, mini-browsers, and wireless browsers) are designed for use on mobile computing devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on a distributed computing platform such as a cloud computing platform. In some embodiments, software modules are hosted on one or more machines in one location. In other embodiments, software modules are hosted on one or more machines in more than one location.

In some embodiments, the platforms, systems, media, and methods disclosed herein include one or more databases, or use of the same. In view of the disclosure provided herein, those of skill in the art will recognize that many databases are suitable for storage and retrieval of medical or substance information. In various embodiments, suitable databases include, by way of non-limiting examples, relational databases, non-relational databases, object-oriented databases, object databases, entity-relationship model databases, associative databases, and XML databases. Further non-limiting examples include SQL, PostgreSQL, My SQL, Oracle, DB2, and Sybase. In some embodiments, a database is internet-based. In further embodiments, a database is web-based. In still further embodiments, a database is cloud computing-based. In a particular embodiment, a database is a distributed database. In other embodiments, a database is based on one or more local computer storage devices.

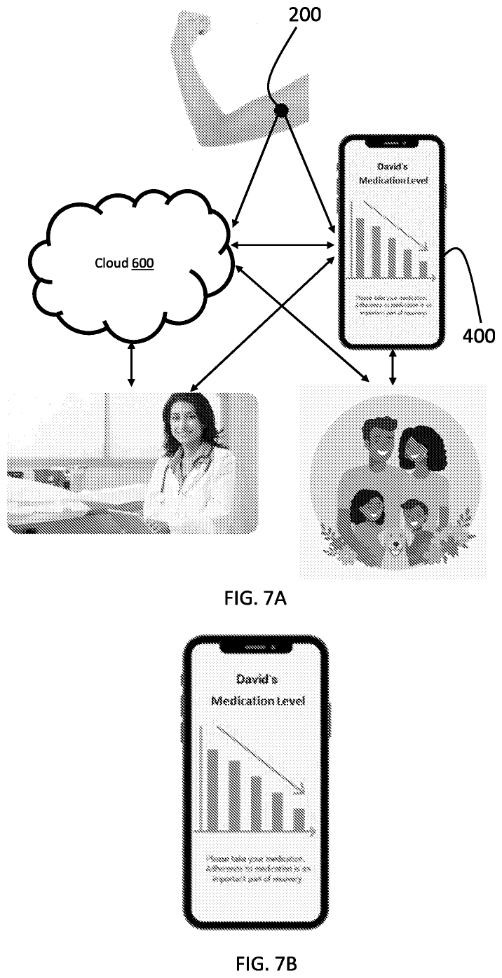

FIG. 7A is a diagram of an exemplary system comprising a sensor 200, a computer system 400, and a cloud-based architecture 600, for the measurement of analytes in the bodily fluid of a subject, according to some embodiments. In some embodiments, a signal generated by the sensor 200 in response to measuring a current or determining the presence, absence, or concentration of an analyte, or both be transmitted by the sensor 200 to the computer system 400 (i.e., mobile phone or smart watch) and the cloud-based architecture 600. In some embodiments, the signal may be further transmitted from the cloud-based architecture 600, to the subject, the subject's family members, and/or the subject's caregivers. In some embodiments, the presence, absence, or concentration of the analyte, the measured current, or both may be determined by the sensor 200, the computer system 400, cloud-based architecture 600, or any combination thereof. In some embodiments, the subject's family members, the subject's caregivers, or both may contact the subject via the cloud-based architecture 600, the computer system 400, or both. In some embodiments, the subject's family members, the subject's caregivers, or both, may contact the patient via the cloud-based architecture 600, the computer system 400, or both, to provide encouragement, updated prescriptions, measurement confirmations, or any combination thereof.

In some embodiments, the presence, absence, or concentration of the analyte, the measured current, or both may be transmitted when the concentration of the substance is larger or smaller than a set threshold from the subject-specific dermal interstitial fluid substance concentration. In some embodiments, the signal may be transmitted periodically. In some embodiments, the signal may be received by the computer system 400 and/or the cloud-based architecture 600, where it may be accessed by either the subject, the subject's caregivers, the subject's clinicians, the subject's family members, or any combination thereof. In some embodiments, the single is transmitted to a smartphone application which may apply additional data processing. In some embodiments, the presence, absence, or concentration of the substance, the measured current, or both may be analyzed to determine patterns, recommend therapeutic improvements, or both. FIG. 7A is an illustration of an exemplary application and user interface, according to some embodiments.

### Processing the Microneedle Array

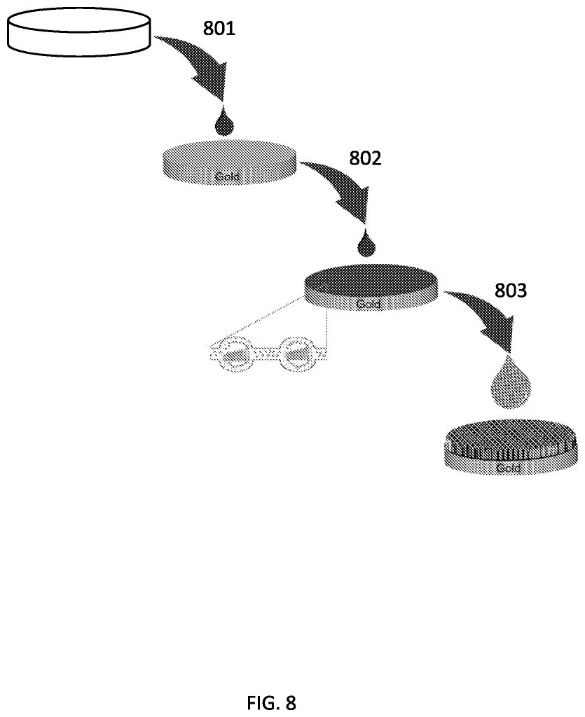

In some embodiments, a method for a processing a microneedle array is provided. FIG. 8 illustrates a method for processing a microneedle array, according to some embodiments. At step 801, each needle of the plurality of needles 212 on the contact surface 211 of the microneedle array 210 is plated with metal or metal alloy. In some embodiments, the metal plating be accomplished by electroplating, vapor phase deposition, atomic layer deposition, and combinations thereof.

In some embodiments, the plurality of needles 212 may be plated with gold, platinum, silver, or combinations thereof. In some embodiments, the plurality of needles 212 may be plated with silver chloride.

At step 802, a polymeric coating (i.e., an electroactive cage 320) is disposed over each metal plated needle of the plurality of needles of the microneedle array. In some embodiments, the polymeric coating may be deposited onto the metal plated plurality of needles using electrodeposition, spin casting, simple dipping, drop casting, and combinations thereof.

In some embodiments, the polymeric coating may comprise an electroactive composite and a stabilizing polymer matrix. In some embodiments, the electroactive coating and the stabilizing polymer matrix may self-assemble into an electro active cage.

In some embodiments, the electroactive composite comprises a polymer selected from polyethyleneimine (PEI), polyethylene (PE), polydimethyl siloxane (PDMS), polytetrafluoroethylene (PTFE), or combinations thereof.

In some embodiments, the stabilizing polymer matrix may comprise an alkyl ammonium salt and a sulfonated tetrafluoroethylene polymer.

In some embodiments, the electroactive composite may be disposed over each of the metal plated needles of the plurality of needles before disposing the stabilizing polymer matrix. In some embodiments, the stabilizing polymer matrix may be disposed over each of the metal plated needles of the plurality of needles before disposing the electroactive composite. In some embodiments, the electroactive composite and the stabilizing polymer matrix are disposed over each of the metal plated needles of the plurality of needles simultaneously. In some embodiments, the disposition of either the electroactive composite or the stabilizing polymer matrix may be performed more than once to form a plurality of polymeric coating layers or to achieve a desired thickness of the polymeric coating. In some embodiments, the electroactive coating and the stabilizing polymer matrix may be combined prior to deposition of the polymeric coating.

In some embodiments, the electroactive composite further comprises MWCNT. In some embodiments, the electroactive composite is prepared by dispersing the MWCNT into a solution to generate a dispersion, and then combining the dispersion with an aqueous solution comprising a polymer. In some embodiments, the solvent used to disperse the MWCNT may be an alcohol, water, a non-polar organic solvent (e.g. hexane), or combinations thereof. In some embodiments, the ratio of alcohol to water in such a solvent is 100:1 or 1:100 or any ratio in between, for example, 100:1, 75:1, 50:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:50, 1:75, and 1:100, to name a few.

In some embodiments, the method may further comprise removing any unbound MWCNT or polymer from each of the needles after deposition of the electroactive composite. In some embodiments, removing any unbound MWCNT or polymer may be accomplished by rinsing the microneedle array with a solvent. In some embodiments, the solvent used to rinse the microneedle array may be an alcohol, water, a non-polar organic solvent (e.g. hexane), or combinations thereof. In some embodiments, the method may further comprise drying the microneedle array after rinsing the microneedle array with the solvent.

At step 803, a hydrogel coating is disposed over the polymer coated needles of the microneedle array. In some embodiments, the hydrogel coating may be an agarose-based hydrogel. In some embodiments, the hydrogel coating may be doped with an electrolyte or a solution containing an electrolyte in order to increase the electrical conductivity of the hydrogel. In some embodiments, the electrolyte may be a metal chloride, a metal sulfate, a metal nitrate, a metal nitrite, or combinations thereof. In some embodiments, the electrolyte may be LiCl, NaCl, KCl, CaCl₂, MgCl₂, or combinations thereof. In some embodiments, the electrolyte may be an alkyl-ammonium salt.

In some embodiments, the hydrogel coating may be deposited onto the metal plated plurality of needles using electrodeposition, spin casting, simple dipping, drop casting, and combinations thereof.

In some embodiments, the method may further comprise pre-treating the metal or metal alloy plated plurality microneedle array prior to depositing the polymeric coating. In some embodiments, pre-treating the microneedle array comprises applying an acid to the metal or metal alloy plated microneedle array. In some embodiments, the pre-treating the microneedle array comprises applying a linear potential between the metal or metal alloy plated microneedle array and a reference electrode. In some embodiments, the pre-treating the microneedle array comprises washing the metal or metal alloy plated microneedle array with de-ionized water, followed by drying the metal or metal alloy plated microneedle array.

### Analyte Detection and Measurement

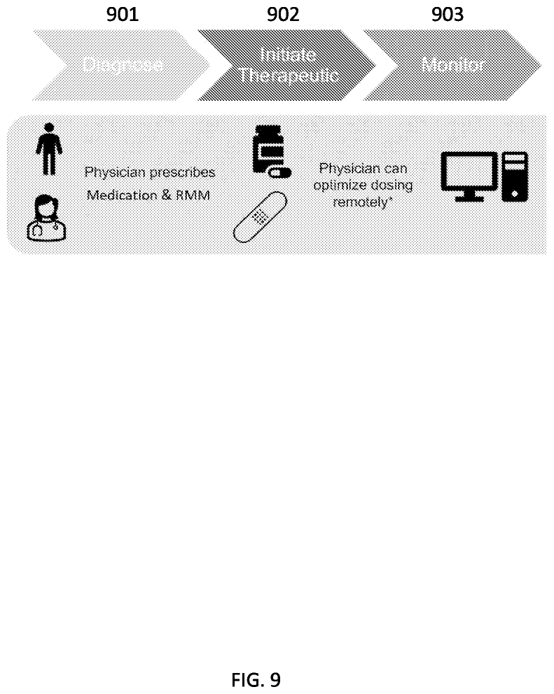

FIG. 9 is a flow chart illustrating an application for the exemplary system for simultaneous measurement of multiple analytes. In one application, the systems described herein may be used in the treatment of a physiological disorder, comprising the steps of diagnosing 901 a subject with a physiological disorder, prescribing 902 a therapeutic regimen for the treatment of the physiological disorder, and monitoring 903 the treatment regimen using the systems and devices described herein. In some embodiments, the physiological disorder may be a substance dependency, a psychiatric condition, an organ disease, or combinations thereof.

In some embodiments, monitoring 903 the treatment regimen using the systems and devices described herein may allow for dosage regulation and/or ensure prescription compliance to enable proper treatment of a physiological disorder.

In some embodiments, monitoring 903 the treatment regimen using the systems and devices described herein may be accomplished by determining the presence or concentration of an analyte in the bodily fluids of a subject.

In some embodiments, determining the presence or concentration of an analyte in the bodily fluids of a subject comprises attaching to the skin of a subject, the sensor described herein, applying a sweeping voltage to the microneedle array of the sensor, measuring a current in response to the applied sweeping voltage, and determining the presence or absence of at least one analyte in the bodily fluid of a subject using the measured current.

In some embodiments, sensor may comprise a working electrode, a reference electrode, a counter electrode and a potentiostat. In some embodiments, the sensor may comprise a working electrode, a combined reference and counter electrode, and a potentiostat. In some embodiments, the sensor may comprise the microneedle array described herein. In some embodiments, the microneedle array may comprise electrically isolated domains, each of which may independently be a working electrode, a reference electrode, or a counter electrode. The working electrode, reference electrode, and counter electrode of the microneedle array may comprise at least one needle of the microneedle array.

In some embodiments, applying a voltage sweep may comprise applying a voltage sweep between a working electrode and a reference electrode using a potentiostat. In some embodiments, measuring a current may comprise measuring a current between a working electrode and a counter electrode in response to applying a sweeping voltage. In some embodiments, determining the presence or absence of at least one analyte in the bodily fluid of a subject may be determined from a current measured between a working electrode and a counter electrode.

In some embodiments, the steps of applying a voltage sweep, measuring a current in response to the voltage sweep and determining the presence or absence of at least one analyte may be repeated at an interval of about 1 minute to about 6-hours. In some embodiments, the steps may be at an interval of about 5 seconds to about 30 second, to about 60 seconds, to about 2 minutes, to about 5 minutes, to about 10 minutes, to about 20 minutes, to about 30 minutes, to about 45 minutes, to about 60 minutes, to about 2 hours, to about 3 hours, to about 4 hours, to about 5 hours, or to about 6 hours. In some embodiments, the steps may be at an interval of no greater than about 6 hours, about 5 hours, about 4 hours, about 3 hours, about 2 hours, about 1 hour, about 45 minutes, about 30 minutes, about 20 minutes, about 10 minutes, about 5 minutes, about 2 minutes, about 1 minute, about 30 seconds.

In some embodiments, the sweeping voltage is applied between the working electrode and the reference electrode. In some embodiments, the sweeping voltage may be about −3.0 V to about +3.0 V. In some embodiments, the sweeping voltage may be about −2.75 V to about +2.75 V, about −2.5 V to about +2.5 V, about −2.0 V to about +2.0 V, about −1.75 V to about +1.75 V, about −1.5 V to about +1.5 V, about −1.25 V to about +1.5 V, about −1.0 V to about +1.5 V, about −0.75 V to about +1.5 V, about −0.5 V to about +1.5 V, about −0.5 V to about +1.25 V, about −0.5 V to about +1.0 V. about −0.5 V to about +0.75 V, or about −0.5 V to about +0.5 V. In some embodiments, the sweeping voltage may be from about −0.7 V to about +1.5 V.

In some embodiments, the voltage sweep may be a single voltage sweep. In some embodiments, the single voltage sweep occurs over a period of about 5 seconds to about 6-hours. In some embodiments, the single voltage sweep occurs over a period of about 5 seconds to about 30 second, to about 60 seconds, to about 2 minutes, to about 5 minutes, to about 10 minutes, to about 20 minutes, to about 30 minutes, to about 45 minutes, to about 60 minutes, to about 2 hours, to about 3 hours, to about 4 hours, to about 5 hours, or to about 6 hours. In some embodiments, the single voltage sweep occurs over a period of no greater than about 6 hours, about 5 hours, about 4 hours, about 3 hours, about 2 hours, about 1 hour, about 45 minutes, about 30 minutes, about 20 minutes, about 10 minutes, about 5 minutes, about 2 minutes, about 1 minute, about 30 seconds.

In some embodiments, the single voltage sweep may either be a single liner voltage sweep or a single staircase voltage sweep. In some embodiments, the voltage sweep is a single staircase voltage sweep.

In some embodiments, the voltage sweep may be a multiple voltage sweep. In some embodiments, the multiple voltage sweep may occur over a period of about 5 seconds to about 6-hours. In some embodiments, the multiple voltage sweep occurs over a period of about 5 seconds to about 30 second, to about 60 seconds, to about 2 minutes, to about 5 minutes, to about 10 minutes, to about 20 minutes, to about 30 minutes, to about 45 minutes, to about 60 minutes, to about 2 hours, to about 3 hours, to about 4 hours, to about 5 hours, or to about 6 hours. In some embodiments, the multiple voltage sweep occurs over a period of no greater than about 6 hours, about 5 hours, about 4 hours, about 3 hours, about 2 hours, about 1 hour, about 45 minutes, about 30 minutes, about 20 minutes, about 10 minutes, about 5 minutes, about 2 minutes, about 1 minute, about 30 seconds.

In some embodiments, the multiple voltage sweep may either be a multiple liner voltage sweep or a multiple staircase voltage sweep. In some embodiments, the voltage sweep is a multiple staircase voltage sweep.

In some embodiments, the method further comprises determining the subject-specific dermal interstitial fluid substance concentration of the substance in the subject comprising: medicating the subject with a known dose of the substance; and determining, with the sensor, a calibration concentration of the substance in the subject; wherein the subject-specific dermal interstitial fluid substance concentration is based on the known dose and the calibration concentration of the substance. In some embodiments, the calibration concentration of the substance is time-dependent.

In some embodiments, the determining the presence or absence of the at least one analyte in the bodily fluids of a subject does not require calibrating the sensor. In some embodiments, the sensor may be calibrated using a calibration data set.

In some embodiments, monitoring the magnitude of the current between the working electrode and the counter electrode (or between the counter electrode and the working electrode) over time may provide a threshold value for determining concentration changes over time.

In some embodiments, the method may further comprise determining the concentration of the analyte in the bodily fluid of the subject using the measured current.

In some embodiments, the method further comprises transmitting a signal from the sensor when either the presence, absence, or concentration is determined, or when the current is measured, or both, providing in-vivo drug concentrations in real-time. Real-time in-vivo drug concentrations reveal information about an individual's unique metabolization rate which may be critical for determining dosing and therapeutic adjustments.

In some embodiments, the analyte is a pharmaceutical, an illicit drug or their respective metabolites. In some embodiments, the pharmaceutical comprises methadone or a methadone metabolite. In some embodiments, the methadone or methadone metabolite is selected from methadone, 2-ethylidene-1,5-dimethyl-3,3-diphenylpyrrolidine (EDDP), methadone-3-glucuronide, methadone-6-glucuronide, and combinations thereof.

In some embodiments, the pharmaceutical comprises buprenorphine or a buprenorphine metabolite. In some embodiments, the buprenorphine or a buprenorphine metabolite is selected from buprenorphine, norbuprenorphine, buprenorphine-3-glucuronide, norbuprenorphine-3-glucuronide, and combinations thereof.

In some embodiments, the pharmaceutical comprises opioids. In some embodiments, the opioids are selected from codeine, dihydrocodeine, fentanyl, carfentanyl, hydromorphone, hydrocodone, meperidine, morphine, oxycodone, oxymorphone, tapentadol, tramadol, and combinations thereof.

In some embodiments, the pharmaceutical comprises anti-cancer drugs or antimetabolites. In some embodiments, the anti-cancer drugs and antimetabolites are selected from methotrexate, gemcitabine, 6-mercaptopurine, fludarabine, cytarabine, pemetrexed, and combinations thereof.

In some embodiments, the pharmaceutical comprises immunosuppressants. In some embodiments, the immunosuppressants are selected from cyclosporines, mycophenolate mofetil, prednisone, tacrolimus, azathioprine, and combinations thereof.

In some embodiments, the pharmaceutical comprises antibiotics. In some embodiments, the antibiotics include aminoglycosides. In some embodiments, the antibiotics are selected from ciprofloxacin, amoxicillin, vancomycin, gentamycin, tetracyclines, and combinations thereof.

In some embodiments, the pharmaceutical comprises anxiolytics. In some embodiments, the anxiolytics are selected from cannabidiol, alprazolam, diazepam, lorazepam, chlordiazepoxide, clonazepam, midazolam, and combinations thereof.

In some embodiments, the pharmaceutical composition is anticonvulsants. In some embodiments, the anticonvulsants are selected from clobazam, gabapentin, pregabalin, oxcarbazepine, phenobarbital, valproic acid, zonisamide, levetiracetam, lamotrigine, lacosamide, topiramate, and combinations thereof.

In some embodiments, the pharmaceutical composition is an Selective Serotonin Reuptake Inhibitor (SSRI). In some embodiments the SSRI is selected from fluoxetine, sertraline, paroxetine, escitalopram, citalopram, and combinations thereof.

In some embodiments, the pharmaceutical composition is a drug with a narrow therapeutic index (NTI). In some embodiments, the NTI is selected from dioxin, digitoxin, flecainide, lithium, phenytoin, rifampicin, theophylline, warfarin, and combinations thereof.

In some embodiments, the pharmaceutical comprises a metal. In some embodiments, the metal is selected from sodium, potassium, iron, cobalt, copper, magnesium, manganese, and combinations thereof.

In some embodiments, the pharmaceutical comprises a hormone or neurotransmitter. In some embodiments, the hormone or neurotransmitter is selected from norepinephrine, epinephrine, testosterone, estrogen, progesterone, cortisol, aldosterol, dopamine, serotonin, acetylcholine, and combinations thereof.

In some embodiments, the pharmaceutical is other metabolites or nutrients. In some embodiments, the other metabolites or nutrients are selected from creatinine, urea, uric acid, glucose, glutamine, lactate, amino acids (i.e., trytophan, tyrosine, phenylalanine, etc.), amino acid metabolites (i.e., 4-ethylphenyl sulfate), vitamins (i.e., E, A, C, D etc.), essential fatty acids (i.e., linoleic acid), ammonia, and combinations thereof.

In some embodiments, the pharmaceutical is a blood constituent. In some embodiments, the blood constituent is selected form insulin, transferrin, insulin-like growth factor I and II, human platelet-derived growth factor I and II, fibroblasts growth factors, tissue specific growth factors, and combinations thereof.

In some embodiments, the pharmaceutical comprises a compound selected from acetaminophen, alfentanil, amiodipine, amitriptyline, amprenavir, atorvastatin, bepridil, buspirone, caffeine, carbamazepine, carvedilol, celecoxib, cerivastatin, chlorpromazine, cisapride, citalopram, clarithromycin, clomipramine, clozapine, corticosteroids, cyclobenzaprine, cyclophosphamide, dapsone, delavirdine, desipramine, dextromethorphan, diclofenac, diltiazem, disopyramide, dofetilide, donepezil, doxorubicin, efavirenz, erythromycin, ethinylestradiol, etoposide, felodipine, finasteride, flecainide, flurbiprofen, fluvastatin, glimepiride, grepafloxacin, haloperidol, ibuprofen, ifosfamide, imipramine, indinavir, irbesartan, isradipine, itraconazole, ketoconazole, lansoprazole, loratadine, losartan, lovastatin, maprotiline, metoprolol, mexiletine, mirtazapine, montelukast, naproxen, nefazodone, nelfinavir, nicardipine, nifedipine, nimodipine, nortriptyline, olanzapine, omeprazole, paclitaxel, paroxetine, perphenazine, pimozide, piroxicam, propafenone, propranolol, propranolol, propofol, quetiapine, quinidine, quinine, rifabutin, repaglinide, riluzole, risperidone, ritonavir, ropinirole, saquinavir, sertraline, sibutamine, sildenafil, simvastatin, sirolimus, sufentanil, tacrine, tacrolimus, tamoxifen, testosterone, theophylline, thoridazine, timolol, tolbutamide, tolterodine, toremifene, torsemide, trazodone, triazolam, troleandomycin, venlafaxine, verapamil, vinblastine, vincristine, zafirlukast, zaleplon, zileuton, zolpidem, and combinations thereof.

### Examples

The invention now being generally described, it will be more readily understood by reference to the following examples which are included merely for purposes of illustration of certain aspects and embodiments of the present invention, and are not intended to limit the invention in any way.

### Example 1. Preparation of a Coated Microneedle Array

An electroactive composite was prepared dispersing multiwalled carbon nanotubes (MWCNT) in an ethanol/water solvent system to form a dispersion. After sonification and centrifugation of the dispersion, to remove any impurities, the dispersion was combined with an aqueous solution of polyethyleneimine (PEI) to form an electroactive composite suspension. The electroactive composite suspension was centrifuged to remove any unbound PEI or MWCNT, the remaining solvent was drained, and the electroactive composite was air dried.

A stabilizing polymer matrix including an alkyl ammonium salt and a NAFION polymer was prepared using a procedure along the lines described by S. Meredith, S. Xu, M. T. Meredith and S. D. Minteer, JoVE (Journal of Visualized Experiments) 2012 Issue 65 Pages e3949.

The electroactive composite and the stabilizing polymer were mixed, and then allowed to self-assemble into an electroactive three-dimensional cage like structure (i.e., electroactive cage).

A platinum or gold coated microneedle array is obtained and electrochemically pretreated using aqueous sulfuric acid, followed by successive cyclic voltammetry sweeps from −0.4 V to +1.6 V at a potential sweep step of 0.05 V/ssec. After the CV sweeps, the metal surface was washed with deionized water and then air dried. The electroactive cage was then drop-cast onto the metal microneedle array.

A hydrogel was prepared by wetting an agarose-based hydrogel with a KCl solution to increase the electrical conductivity of the hydrogel. The doped hydrogel is then applied to the microneedle array to protect the electroactive coating both mechanically and chemically, from surface deposition of redox byproducts (biofouling).

### Example 2. Analyte Detection

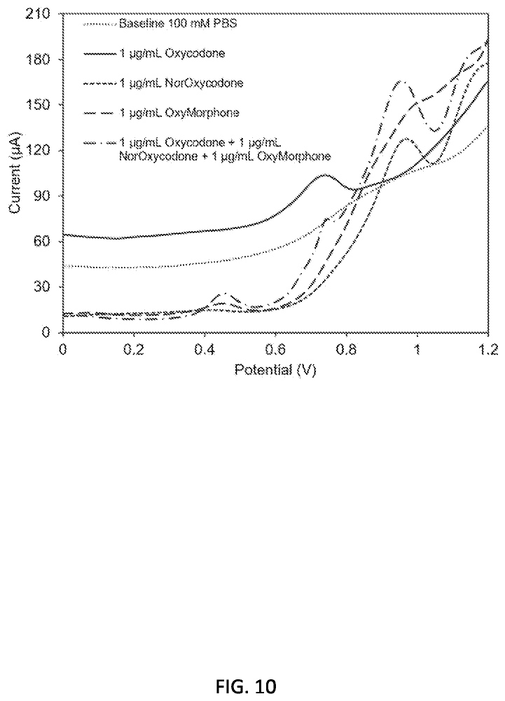

A device according to this disclosure comprising a microneedle sensor array may be used to monitor concentrations of oxymorphone, oxycodone, and noryoxycodone. FIG. 10 shows a Differential Pulse Voltammetry (DPV) graph of exemplary current measurements under applied voltages from 0 to 1.2V for oxymorphone, oxycodone, and noryoxycodone. As shown therein, the current peak of about 30 μA at about 0.45V for oxymorphone is distinctive from the current peak of about 100 μA at about 0.75V for oxycodone, which is also distinctive from the current peak of about 165 μA at about 0.95V for noroxycodone.

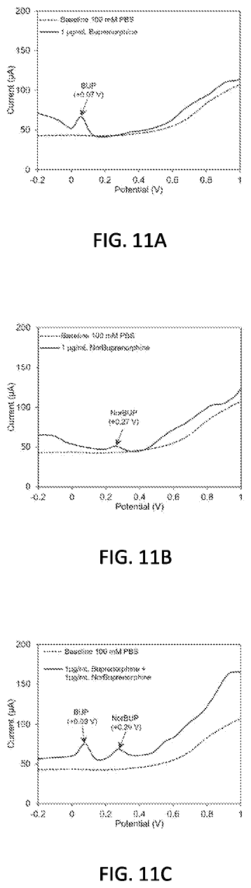

A device according to this disclosure comprising a microneedle sensor array may be used to monitor concentrations of buprenorphine, norbuprenorphine, and both buprenorphine and norbuprenorphine. FIGS. 11A-11C show a DPV graph of exemplary current measurements under applied voltages from −0.2 to 1.0V for a patient taking 1 μg/ml of buprenorphine, 1 μg/ml of norbuprenorphine, and both 1 μg/ml of buprenorphine and 1 μg/ml of norbuprenorphine, respectively, against a baseline unmedicated current measurement. As shown in FIG. 11A, the buprenorphine patient displays a measured current peak at about 0.07V. Further, per FIG. 11B, the norbuprenorphine patient displays a measured current peak at about 0.27V. When the patient receives both buprenorphine and norbuprenorphine, the current peaks of 0.09V for buprenorphine and 0.29V for norbuprenorphine are maintained, as illustrated in FIG. 11C. As such, the methods and devices herein enable the quantification of multiple substances in a single voltage sweep.

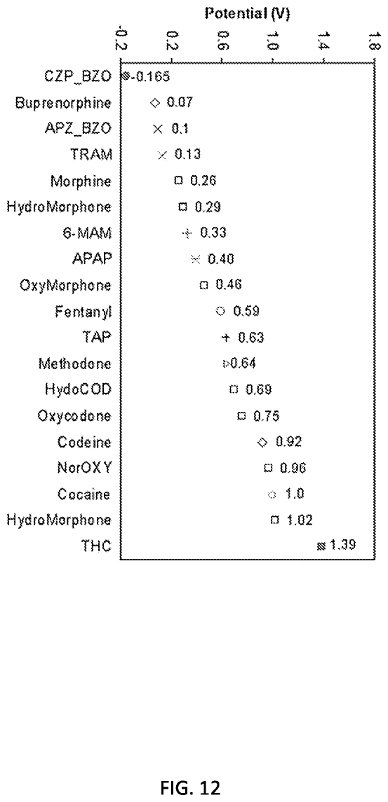

A device according to this disclosure comprising a microneedle sensing array may be used to monitor concentrations of various substances. FIG. 12 summarizes exemplary peak voltages for different substances.

### Example 3. Clinical Applications

A device according to this disclosure comprising a microneedle sensor array is used to monitor concentrations of medications where there is a clinical need to verify compliance (e.g., antidepressants and anti-seizure drugs, and/or drugs that have very strict titration/dosing requirements, due to having a narrow therapeutic window such as immunosuppressants, antibiotics, or blood thinners).

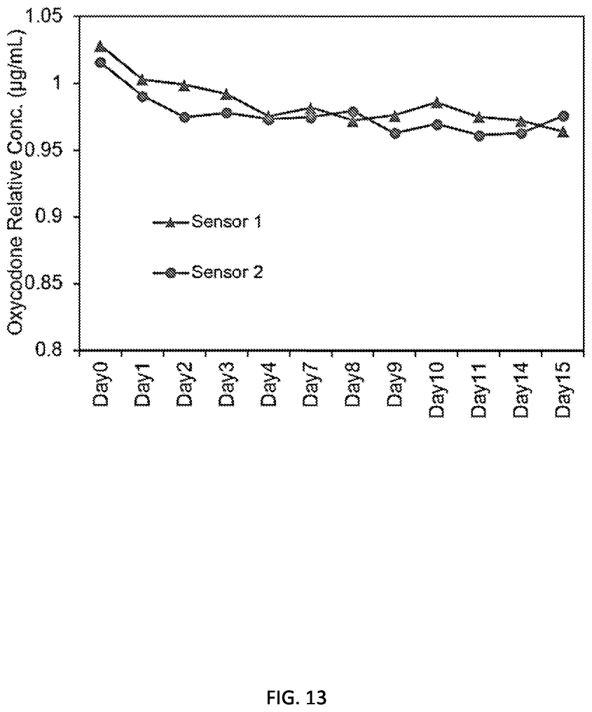

A physician may diagnose a patient as having an opioid addiction. The physician may provide a prescription of buprenorphine and the microneedle sensor described herein. Upon wearing the microneedle sensor and initiating doses of buprenorphine, the physician can view the measured concentration of the substance and optimize the patients dosage accordingly. The physician can continue to monitor the dermal interstitial fluid concentrations of the patient to ensure compliance and update the patient's prescription as necessary. Example data for this type of monitoring are shown in FIG. 13.

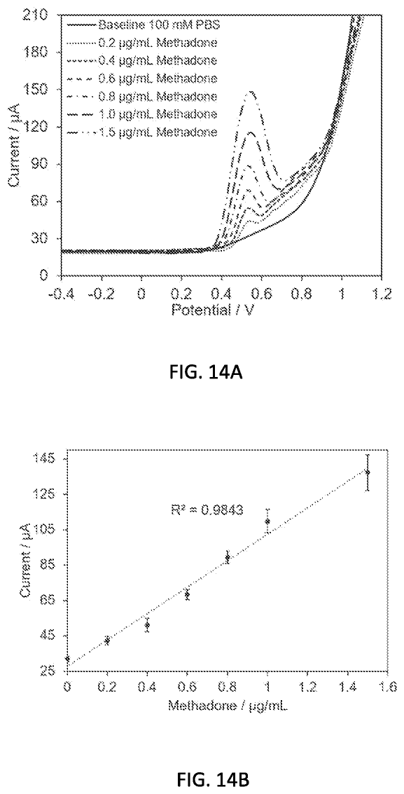

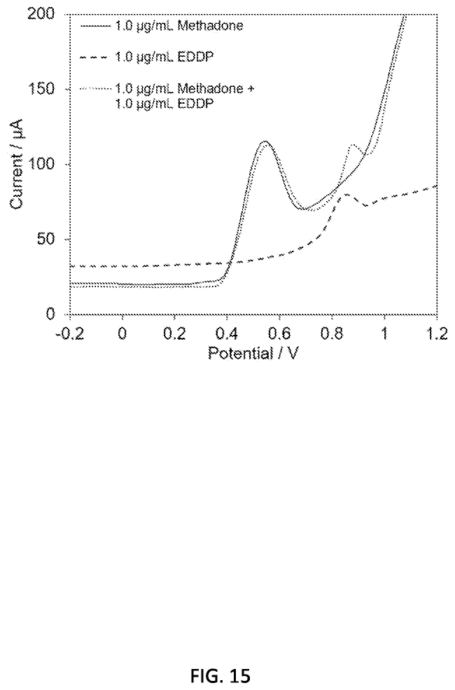

A device according to this disclosure comprising a microneedle sensor array is used to monitor concentrations of methadone and its primary metabolite, 2-ethylidene-1,5-dimethyl-3,3-diphenylpyrrolidine (EDDP) in the dermal interstitial fluid of a subject undergoing methadone treatment. The monitored concentration is reported by the device to a treating physician who uses it to validate that the patient has taken their prescribed doses. Example data and an example calibration curve for this type of monitoring are shown in FIGS. 14A, 14B and 15.

Additionally, the physician may use the data reported by the microneedle sensor array to inform therapeutic dosing decisions by identifying Peak-trough points, as well as pharmacokinetics and -dynamics which are personalized to the specific patient.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

While the foregoing disclosure has been described in some detail for purposes of clarity and understanding, it will be clear to one skilled in the art from a reading of this disclosure that various changes in form and detail can be made without departing from the true scope of the disclosure. For example, all the techniques and apparatus described above can be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

1-76. (canceled)

77. A method of determining the presence or absence of a at least one substance in a bodily fluid of a subject, the method comprising:
attaching the microneedle sensor to the skin of the subject, wherein the microneedle sensor comprises a microneedle array;
applying a sweeping voltage to the microneedle array;
measuring a current, with the microneedle sensor, in response to the sweeping voltage; and determining the presence of absence of the at least one substance in the bodily fluid of the subject using the measured current.

78. The method of claim 77, further comprising determining the concentration of the at least one substance in the bodily fluid of the subject using the measured current.

79. The method of claim 77, wherein the bodily fluid is dermal interstitial fluid.

80. The method of claim 77, wherein determining the presence or absence of the at least one substance in a bodily fluid of a subject does not require an enzymatic reaction involving the at least one substance; or
wherein determining the presence or absence of the at least one substance in a bodily fluid of a subject does not require calibrating the microneedle sensor; or
optionally further comprising repeating the steps of applying, measuring, and determining on an interval between about 1 minute and about 6 hours; or
wherein the microneedle sensor is attached to the skin for about 1 day to about 30 days; or
wherein the microneedle sensor is attached to the skin for at least about 30 days without substantial biofouling; or
wherein the sweeping voltage is from about −0.7 V to about +1.5 V; or
wherein the sweeping voltage comprises a single staircase voltage sweep; or
wherein the sweeping voltage comprises a single staircase voltage sweep and wherein the single staircase voltage sweep occurs over a period of about 5 seconds to about 5 minutes; or
wherein the sweeping voltage comprises multiple staircase voltage sweeps; or
wherein the sweeping voltage comprises multiple staircase voltage sweeps and wherein the multiple staircase voltage sweeps occur over a period of about 5 seconds to about 5 minutes; or
wherein measuring the current comprises generating at least one voltammogram; or
wherein measuring the current comprises generating at least one voltammogram and wherein the presence of absence of the at least one substance in the bodily fluid of the subject is determined using the at least one voltammogram; or
wherein measuring the current comprises generating at least one voltammogram and wherein the presence of absence of the at least one substance in the bodily fluid of the subject is determined using the at least one voltammogram and wherein the presence of absence of the at least one substance in the bodily fluid of the subject is determined using at least one voltage peak of the at least one voltammogram; or
wherein the at least one substance in the bodily fluid of the subject is present when the measured current is above a threshold; or
wherein determining the presence of absence of the at least one substance in the bodily fluid of the subject comprises determining the concentration of the at least one substance; or
wherein determining the presence of absence of the at least one substance in the bodily fluid of the subject comprises determining the concentration of the at least one substance and wherein the at least one substance in the bodily fluid of the subject is present when the concentration of the at least one substance is above a threshold; or
wherein determining the presence or absence of the at least one substance in the bodily fluid of the subject comprises determining the presence or absence of at least two substances in the bodily fluid of the subject concurrently using the measured current; or
wherein the measured concentration of the substance is time-dependent; or
wherein the at least one substance is a pharmaceutical compound or a metabolite of the pharmaceutical compound; or
wherein the at least one substance is a pharmaceutical compound or a metabolite of the pharmaceutical compound and wherein the pharmaceutical compound or a metabolite of the pharmaceutical compound comprises methadone, 2-ethylidene-1,5-dimethyl-3,3-diphenylpyrrolidine (EDDP), methadone-3-glucuronide, methadone-6-glucuronide, buprenorphine or a buprenorphine metabolite is selected from buprenorphine, norbuprenorphine, buprenorphine-3-glucuronide, norbuprenorphine-3-glucuronide, codeine, dihydrocodeine, fentanyl, carfentanyl, hydromorphone, hydrocodone, meperidine, morphine, oxycodone, oxymorphone, tapentadol, tramadol, methotrexate, gemcitabine, 6-mercaptopurine, fludarabine, cytarabine, pemetrexed, cyclosporines, mycophenolate mofetil, prednisone, tacrolimus, azathioprine, ciprofloxacin, amoxicillin, vancomycin, gentamycin, tetracyclines, cannabidiol, alprazolam, diazepam, lorazepam, chlordiazepoxide, clonazepam, midazolam, clobazam, gabapentin, pregabalin, oxcarbazepine, phenobarbital, valproic acid, zonisamide, levetiracetam, lamotrigine, lacosamide, topiramate, fluoxetine, sertraline, paroxetine, escitalopram, citalopram, dioxin, digitoxin, flecainide, lithium, phenytoin, rifampicin, theophylline, warfarin, sodium, potassium, iron, cobalt, copper, magnesium, manganese, norepinephrine, epinephrine, testosterone, estrogen, progesterone, cortisol, aldosterol, dopamine, serotonin, acetylcholine, creatinine, urea, uric acid, glucose, glutamine, lactate, amino acids, amino acid metabolites, vitamins, essential fatty acids, ammonia, insulin, transferrin, insulin like growth factor I and II, human platelet-derived growth factor I, human platelet-derived growth factor II, fibroblasts growth factors, tissue specific growth factors, acetaminophen, alfentanil, amiodipine, amitriptyline, amprenavir, atorvastatin, bepridil, buspirone, caffeine, carbamazepine, carvedilol, celecoxib, cerivastatin, chlorpromazine, cisapride, citalopram, clarithromycin, clomipramine, clozapine, corticosteroids, cyclobenzaprine, cyclophosphamide, dapsone, delavirdine, desipramine, dextromethorphan, diclofenac, diltiazem, disopyramide, dofetilide, donepezil, doxorubicin, efavirenz, erythromycin, ethinylestradiol, etoposide, felodipine, finasteride, flecainide, flurbiprofen, fluvastatin, glimepiride, grepafloxacin, haloperidol, ibuprofen, ifosfamide, imipramine, indinavir, irbesartan, isradipine, itraconazole, ketoconazole, lansoprazole, loratadine, losartan, lovastatin, maprotiline, metoprolol, mexiletine, mirtazapine, montelukast, naproxen, nefazodone, nelfmavir, nicardipine, nifedipine, nimodipine, nortriptyline, olanzapine, omeprazole, paclitaxel, paroxetine, perphenazine, pimozide, piroxicam, propafenone, propranolol, propranolol, propofol, quetiapine, quinidine, quinine, rifabutin, repaglinide, riluzole, risperidone, ritonavir, ropinirole, saquinavir, sertraline, sibutamine, sildenafil, simvastatin, sirolimus, sufentanil, tacrine, tacrolimus, tamoxifen, testosterone, theophylline, thoridazine, timolol, tolbutamide, tolterodine, toremifene, torsemide, trazodone, triazolam, troleandomycin, venlafaxine, verapamil, vinblastine, vincristine, zafirlukast, zaleplon, zileuton, zolpidem, and combinations thereof.

81. The method of claim 77, wherein the microneedle array comprises at least one working electrode, at least one reference electrode, at least one counter electrode, and a potentiostat; or
wherein the at least one working electrode, at least one reference electrode, and the at least one counter electrode each independently comprise at least one microneedle of the microneedle array; or
wherein applying the sweeping voltage to the microneedle array comprises applying the sweeping voltage between the at least one working electrode and the at least one reference electrode; or
wherein measuring the current comprises generating at least one voltammogram; or
wherein measuring the current comprises generating at least one voltammogram and wherein the presence of absence of the at least one substance in the bodily fluid of the subject is determined using the at least one voltammogram; or
wherein measuring the current comprises generating at least one voltammogram and wherein the presence of absence of the at least one substance in the bodily fluid of the subject is determined using the at least one voltammogram and wherein the presence of absence of the at least one substance in the bodily fluid of the subject is determined using at least one voltage peak of the at least one voltammogram; or
wherein the at least one substance in the bodily fluid of the subject is present when the measured current is above a threshold; or
wherein determining the presence of absence of the at least one substance in the bodily fluid of the subject comprises determining the concentration of the at least one substance; or
wherein determining the presence of absence of the at least one substance in the bodily fluid of the subject comprises determining the concentration of the at least one substance and wherein the at least one substance in the bodily fluid of the subject is present when the concentration of the at least one substance is above a threshold; or
wherein determining the presence or absence of the at least one substance in the bodily fluid of the subject comprises determining the presence or absence of at least two substances in the bodily fluid of the subject concurrently using the measured current; or
wherein the measured concentration of the substance is time-dependent; or
wherein the at least one substance is a pharmaceutical compound or a metabolite of the pharmaceutical compound; or
wherein the at least one substance is a pharmaceutical compound or a metabolite of the pharmaceutical compound and wherein the pharmaceutical compound or a metabolite of the pharmaceutical compound comprises methadone, 2-ethylidene-1,5-dimethyl-3,3-diphenylpyrrolidine (EDDP), methadone-3-glucuronide, methadone-6-glucuronide, buprenorphine or a buprenorphine metabolite is selected from buprenorphine, norbuprenorphine, buprenorphine-3-glucuronide, norbuprenorphine-3-glucuronide, codeine, dihydrocodeine, fentanyl, carfentanyl, hydromorphone, hydrocodone, meperidine, morphine, oxycodone, oxymorphone, tapentadol, tramadol, methotrexate, gemcitabine, 6-mercaptopurine, fludarabine, cytarabine, pemetrexed, cyclosporines, mycophenolate mofetil, prednisone, tacrolimus, azathioprine, ciprofloxacin, amoxicillin, vancomycin, gentamycin, tetracyclines, cannabidiol, alprazolam, diazepam, lorazepam, chlordiazepoxide, clonazepam, midazolam, clobazam, gabapentin, pregabalin, oxcarbazepine, phenobarbital, valproic acid, zonisamide, levetiracetam, lamotrigine, lacosamide, topiramate, fluoxetine, sertraline, paroxetine, escitalopram, citalopram, dioxin, digitoxin, flecainide, lithium, phenytoin, rifampicin, theophylline, warfarin, sodium, potassium, iron, cobalt, copper, magnesium, manganese, norepinephrine, epinephrine, testosterone, estrogen, progesterone, cortisol, aldosterol, dopamine, serotonin, acetylcholine, creatinine, urea, uric acid, glucose, glutamine, lactate, amino acids, amino acid metabolites, vitamins, essential fatty acids, ammonia, insulin, transferrin, insulin like growth factor I and II, human platelet-derived growth factor I, human platelet-derived growth factor II, fibroblasts growth factors, tissue specific growth factors, acetaminophen, alfentanil, amiodipine, amitriptyline, amprenavir, atorvastatin, bepridil, buspirone, caffeine, carbamazepine, carvedilol, celecoxib, cerivastatin, chlorpromazine, cisapride, citalopram, clarithromycin, clomipramine, clozapine, corticosteroids, cyclobenzaprine, cyclophosphamide, dapsone, delavirdine, desipramine, dextromethorphan, diclofenac, diltiazem, disopyramide, dofetilide, donepezil, doxorubicin, efavirenz, erythromycin, ethinylestradiol, etoposide, felodipine, finasteride, flecainide, flurbiprofen, fluvastatin, glimepiride, grepafloxacin, haloperidol, ibuprofen, ifosfamide, imipramine, indinavir, irbesartan, isradipine, itraconazole, ketoconazole, lansoprazole, loratadine, losartan, lovastatin, maprotiline, metoprolol, mexiletine, mirtazapine, montelukast, naproxen, nefazodone, nelfmavir, nicardipine, nifedipine, nimodipine, nortriptyline, olanzapine, omeprazole, paclitaxel, paroxetine, perphenazine, pimozide, piroxicam, propafenone, propranolol, propranolol, propofol, quetiapine, quinidine, quinine, rifabutin, repaglinide, riluzole, risperidone, ritonavir, ropinirole, saquinavir, sertraline, sibutamine, sildenafil, simvastatin, sirolimus, sufentanil, tacrine, tacrolimus, tamoxifen, testosterone, theophylline, thoridazine, timolol, tolbutamide, tolterodine, toremifene, torsemide, trazodone, triazolam, troleandomycin, venlafaxine, verapamil, vinblastine, vincristine, zafirlukast, zaleplon, zileuton, zolpidem, and combinations thereof.

82. The method of claim 77, further comprising transmitting, with the microneedle sensor, a signal generated using the determined presence or absence of the at least one substance in the bodily fluid of the subject, the measured current, or both, to one or more of an external device, system or network.

83. The method of claim 78, further comprising transmitting, with the microneedle sensor, a signal generated using the determined concentration of the at least one substance in the bodily fluid of the subject.

84. The method of claim 82, wherein the signal is transmitted periodically at about 1 min to about 6-hour intervals; or
wherein the signal is transmitted to the one or more external device, system, or network when the absence of the at least one substance is determined; or
where the signal is transmitted to the one or more external device, system, or network when the concentration of the at least one substance is below a threshold concentration.

85. A sensor device for determining the presence or absence of at least one substance in a bodily fluid of a subject, the device comprising
a body configured to attach the skin of the subject;
a microneedle array coupled to the body; and
a control module disposed within the body, the control module comprising a potentiostat coupled to the microneedle array, a processor coupled to the potentiostat, and a memory coupled to the processor,
wherein the memory comprises instructions for the processor to perform the method of claim 77.

86. A sensor device for determining the presence or absence of at least one substance in a bodily fluid of a subject, the device comprising
a body configured to attach the skin of the subject;
a microneedle array coupled to the body; and
a control module disposed within the body, the control module comprising a potentiostat coupled to the microneedle array, a processor coupled to the potentiostat, and a memory coupled to the processor,
wherein the memory comprises instructions for the processor to:
apply a sweeping voltage to the microneedle array;
measure a current in response to applying the sweeping voltage; and
determine the presence or absence of the at least one substance in the bodily fluid of the subject using the measured current.

87. The device of claim 86, wherein the body comprises an adhesive; or
wherein at least one microneedle of the microneedle array is plated with gold, platinum, silver, silver chloride, or combinations thereof; or
wherein at least one microneedle of the microneedle array is coated with an electrode coating; or
wherein at least one microneedle of the microneedle array is coated in an electrode coating and wherein the electrode coating comprises a quaternary ammonium, sulfonated tetrafluoroethylene, polyethyleneimine-functionalized carbon nanotube, or combinations thereof; or
wherein the microneedle array comprises at least one working electrode and at least one reference electrode; or
wherein the microneedle array comprises at least one working electrode and at least one reference electrode and wherein the sweeping voltage is applied between the at least one working electrode and the at least one reference electrode; or
wherein the device does not comprise an enzymatic layer; or
wherein the device does not comprise a microfluidic element, microfluidic channel, or microfluidic pump; or
wherein the memory further comprises instructions for the processor to determine the presence or absence of at least two substances in the bodily fluid of the subject concurrently using the measured current; or
wherein sensor is configured to be attached to the skin for about 1 day to about 30 days; or
wherein the sensor is configured to be attached to the skin for at least about 30 days without biofouling; or
further comprising a receiver coupled to the processor; or
wherein the device further comprises an alarm configured to generate an alert to a user based on the determined presence or absence of the at least one substance, the measured current, or both; or
wherein the device further comprises an alarm configured to generate an alert to a caregiver based on the determined presence or absence of the at least one substance, the measured current, or both; or
wherein the device further comprises an alarm configured to generate an alert to a caregiver based on the determined presence or absence of the at least one substance, the measured current, or both and wherein a caregiver is selected from the group consisting of physician, clinician, pharmacy, government enforcement agency or combinations thereof.

88. The device of claim 86, further comprising a transmitter coupled to the processor.

89. The device of claim 88, wherein the transmitter is configured to transmit a signal based on the determined presence or absence of the at least one substance, the measured current, or both, to one or more of an external device, system, or network; or
wherein the signal is based on a measured concentration of the at one substance; or
wherein the memory further comprises instructions to transmit the signal periodically at about 1 min to about 6-hour intervals; or
wherein the one or more of an external device, system, or network comprises one or more of a smart phone, tablet computer, personal computer, gaming console, workstation, network computer, or network server; or
wherein the one or more of an external device, system, or network comprises a memory comprising instructions for the one or more of an external device, system, or network to operate an application including one or more of a measurement display or a user control for the sensor device; or
wherein the one or more of an external device, system, or network comprises a memory comprising instructions for the one or more of an external device, system, or network to operate an application including one or more of a measurement display or a user control for the sensor device and wherein the application is configured to provide an alert to a user based on the determined presence or absence of the at least one substance in the bodily fluid of a subject; or
wherein the one or more of an external device, system, or network comprises a memory comprising instructions for the one or more of an external device, system, or network to operate an application including one or more of a measurement display or a user control for the sensor device and wherein the application is configured to provide an alert to a caregiver based on the determined presence or absence of the at least one substance in the bodily fluid of a subject; or
wherein the one or more of an external device, system, or network comprises a memory comprising instructions for the one or more of an external device, system, or network to operate an application including one or more of a measurement display or a user control for the sensor device and wherein the application is configured to provide an alert to a caregiver based on the determined presence or absence of the at least one substance in the bodily fluid of a subject and wherein a caregiver is selected from the group consisting of physician, clinician, pharmacy, government enforcement agency or combinations thereof; or
wherein the device further comprises an alarm configured to generate an alert to a user based on the determined presence or absence of the at least one substance, the measured current, or both; or
wherein the device further comprises an alarm configured to generate an alert to a caregiver based on the determined presence or absence of the at least one substance, the measured current, or both; or
wherein the device further comprises an alarm configured to generate an alert to a caregiver based on the determined presence or absence of the at least one substance, the measured current, or both and wherein a caregiver is selected from the group consisting of physician, clinician, pharmacy, government enforcement agency or combinations thereof.

90. A system for determining the presence or absence of at least one substance in a bodily fluid of a subject, the system comprising:
an external device, system, or network; and
the device of claim 86,
wherein the device further comprises a transmitter coupled to a processor of said device, the transmitter being configured to transmit a signal based on the determined presence or absence of the at least one substance, the measured current, or both to the external device, system, or network, and
wherein the external device, system, or network comprises a memory comprising instructions for the external device, system, or network to operate an application including a measurement display or a user control for the device.

91. A microneedle array comprising:
a plurality of microneedles;
a metal or metal alloy plating for the plurality of microneedles; and
an electroactive cage material deposited over the metal plating, wherein the electroactive cage material comprises, (i) an electroactive composite comprising multiwall carbon nanotubes and a polyethyleneimine polymer; (ii) a stabilizing polymer matrix embedding and surrounding the electroactive composite; and (iii) a hydrogel coating provided over the electroactive cage.

92. The array of claim 91, wherein the metal plating comprises gold, platinum, silver, silver chloride, or combinations thereof; or
wherein the stabilizing polymer matrix comprises an alkyl ammonium salt and a sulfonated tetrafluoroethylene polymer; or
wherein the hydrogel coating comprises an agarose hydrogel; or
wherein the hydrogel coating is doped with potassium chloride; or
wherein the hydrogel coating is configured to protect the electroactive cage material from the deposition of molecular redox byproducts.

93. A method of processing a microneedle array, the method comprising:
plating as metal or metal alloy onto a plurality of microneedles;
depositing an electroactive cage material over the metal coating, the electroactive cage material comprising (i) an electroactive composite comprising multiwall carbon nanotubes and a polyethyleneimine polymer; (ii) a stabilizing polymer matrix bound to the electroactive composite; and
providing a hydrogel coating over the electroactive cage material on the plurality of microneedles.

94. The method of claim 93, wherein the metal plating comprises gold, platinum, silver, silver chloride, and combinations thereof; or
further comprising pre-treating the metal or metal alloy plated plurality of microneedles prior to depositing the electro active cage material; or
further comprising pre-treating the metal or metal alloy plated plurality of microneedles prior to depositing the electro active cage material and wherein pre-treating the metal or metal alloy plated plurality of microneedles comprises one or more of the following: applying an acid to the metal or metal alloy plated plurality of microneedles; applying a linear potential sweep between the metal or metal alloy plated plurality of microneedles and a reference electrode; washing the metal or metal alloy plated plurality of microneedles with de-ionized water; and drying the metal or metal alloy plated plurality of microneedles; or
further comprising combining the electroactive composite and the stabilizing polymer matrix and allowing the electroactive composite to self-assemble into the electroactive cage material; or
wherein the stabilizing polymer matrix comprises an alkyl ammonium salt and a sulfonated tetrafluoroethylene polymer; or

95. The method of claim 93, further comprising producing the electroactive cage by dispersing the multiwall carbon nanotubes into a solution to generate a dispersion and combining the dispersion with an aqueous solution comprising the polyethyleneimine polymer.

96. The method of claim 95, further comprising removing unbound multiwall carbon nanotubes and polyethyleneimine polymer; or
further comprising removing unbound multiwall carbon nanotubes and polyethyleneimine polymer and further comprising drying the composite after the unbound multiwall carbon nanotubes and polyethyleneimine polymer is removed; or
further comprising combining the electroactive composite and the stabilizing polymer matrix and allowing the electroactive composite to self-assemble into the electroactive cage material; or
wherein the stabilizing polymer matrix comprises an alkyl ammonium salt and a sulfonated tetrafluoroethylene polymer; or
wherein depositing the electroactive cage material over the metal coating comprises drop-casting the electroactive cage material; or
wherein the hydrogel coating comprises an agarose hydrogel; or
wherein the hydrogel coating is doped with potassium chloride.
